(19) United States
(12) Patent Application Publication
(10) Pub. No.: US 2025/0206775 A1
(43) Pub. Date: Jun. 26, 2025

(54) METHODS OF ISOLATING POLYPEPTIDES

(71) Applicant: BRISTOL-MYERS SQUIBB COMPANY, PRINCETON, NJ (US)

(72) Inventors: Elizabeth BIGELOW, Bolton, MA (US); Yuanli SONG, Acton, MA (US)

(73) Assignee: BRISTOL-MYERS SQUIBB COMPANY, PRINCETON, NJ (US)

(21) Appl. No.: 18/848,359

(22) PCT Filed: Mar. 17, 2023
(86) PCT No.: PCT/US2023/064661

§ 371 Date: Sep. 18, 2024

Related U.S. Application Data
(60) Provisional application No. 63/321,531, filed on Mar. 18, 2022.

(51) Int. Cl. C07K 1/36; G01N 33/68

(52) U.S. Cl. CPC C07K 1/36; G01N 33/6803; G01N 2440/00

(57) ABSTRACT

The present disclosure is directed methods of isolating and/or purifying a species of a protein, comprising contacting a mixture comprising the species and one or more impurities with two or more chromatography columns in a continuous operation mode. In some aspects, the species of the protein is a charge variant.

### CROSS-REFERENCE TO RELATED APPLICATIONS

This application claims priority benefit of U.S. Provisional Application No. 63/321,531, filed Mar. 18, 2022, which is herein incorporated by reference in its entirety.

### FIELD

The present application relates to the field of protein isolation and purification using two or more chromatography columns.

### BACKGROUND

Therapeutic proteins, particularly monoclonal antibodies (mAbs), are subject to various post-translational modifications (PTMs), such as oxidization, deamidation, glycosylation, and lysine truncation. Some of these modifications cause protein charge variants. Characterization and analysis of charge variants for therapeutic proteins is required to ensure that the quality of the drug product is not impacted. However, protein purification and isolation on a large scale can be costly and time consuming. Traditional methods of separating species of a particular protein rely on HPLC and/or FPLC, methods which can take weeks to months to generate substantial product for further analysis and/or use. Traditional methods typically force a decision to have either high productivity or high purity, but both are not usually achieved simultaneously.

As such, there remains a need in the field of protein purification for methods that maximize yield and purity while reducing the time required to generate the purified sample.

### BRIEF SUMMARY

Some aspects of the present disclosure are directed to methods of isolating a species of a protein from a mixture comprising the species and one or more impurities, comprising contacting the mixture with two or more chromatography columns in a continuous operation mode.

Some aspects of the present disclosure are directed to methods of increasing purity and/or yield of a species of a protein from a mixture comprising the species and one or more impurities, comprising contacting the mixture with two or more chromatography columns in a continuous operation mode.

Some aspects of the present disclosure are directed to methods for enriching a species of a protein for analytical characterization, comprising: (a) separating the species of the protein from a mixture comprising the species and one or more impurities, comprising contacting the mixture with two or more chromatography columns in a continuous operation mode in a chromatographic separation system; and (b) subjecting the species from (a) to analytical characterization.

Some aspects of the present disclosure are directed to methods for conducting analytical characterization of a species of a protein, comprising: (a) separating the species of the protein from a mixture comprising the species and one or more impurities, comprising contacting the mixture with two or more chromatography columns in a continuous operation mode in a chromatographic separation system; and (b) conducting analytical characterization of the species from (a).

In some aspects, the analytical characterization is conducted by a HPLC system, capillary isoelectric focusing (cIEF) gel electrophoresis, Imaged Capillary Isoelectric Focusing (iCIEF), cation exchange chromatography (CEX), anion exchange chromatography (AEX), MFI, SEC-MALS, SEC, or mass spectrometry. In some aspects, the method yields an increased purity and/or an increased yield of the species of the protein compared to an HPLC or an FPLC.

In some aspects, the two or more chromatography columns enrich the species.

In some aspects, the method further comprises loading the mixture to the first chromatography column. In some aspects, the loaded mixture passes through the first chromatography column and is separated into an enrichment species comprising the species and a discard species (“enrichment stage I”). In some aspects, the enrichment species passes through the second column and the discard species is discarded after the first chromatography column (“enrichment stage II”).

In some aspects, the method further comprises re-equilibrating the first chromatography column.

In some aspects, the method further comprises contacting the enrichment species with the first chromatography column. In some aspects, the method further comprises loading an additional mixture to the first column, wherein the additional mixture comprises the species and one or more impurities. In some aspects, the additional mixture is added at the same time the enrichment species is added to the first chromatography column. In some aspects, the additional mixture is added after the enrichment species is added to the first chromatography column, and before the enrichment species passes through the first chromatography column.

In some aspects, the enrichment stages I and II are repeated at least twice, at least three times, at least four times, at least five times, at least six times, at least seven times, at least eight times, at least nine times, or at least ten times.

In some aspects, the method further comprises a depletion stage. In some aspects, the depletion stage comprises contacting the enrichment species with the first chromatography column in the absence of an additional mixture. In some aspects, the depletion stage further comprises passing the enrichment species through the first chromatography column and separating the species from one or more impurities. In some aspects, the depletion stage further comprises passing the enrichment species through the second chromatography column, separating the species from one or more impurities.

In some aspects, the method further comprises eluting the species. In some aspects, the method results in at least 88%, 89%, 90%, 91%, 92%, 93%, 95%, 96%, 97%, 98%, 99%, or 100% pure species of the protein.

In some aspects, the concentration of the species eluted is at least about 1.5, at least about 1.6, at least about 1.7, at least about 1.8, at least about 1.9, at least about 2.0, at least about 2.5, at least about 3.0, at least about 3.5, at least about 4.0, at least about 4.5, at least about 5.0, at least about 5.5, at least about 6.0, at least about 6.5, at least about 7.0, at least about 7.5, at least about 8.0, at least about 8.5, at least about 9.0, at least about 9.5, or at least about 10.0 fold higher than the concentration of the species in the mixture.

In some aspects, one or more of the chromatography columns comprise a salt gradient, a pH gradient, or both. In some aspects, the salt gradient comprises a sodium chloride gradient. In some aspects, the salt gradient comprises with or without the salt. In some aspects, the concentration of the salt is between about 50 mM and about 600 mM, between about 100 mM and about 550 mM, between about 150 mM and about 500 mM, between about 200 mM and about 450 mM, between about 250 mM and about 400 mM, between about 100 mM and about 400 mM, between about 100 mM and about 350 mM, between about 100 mM and about 300 mM, between about 100 mM and about 250 mM, between about 300 mM and about 600 mM, between about 350 mM and about 550 mM, between about 400 mM and about 500 mM, or between about 350 mM and about 450 mM. In some aspects, the concentration of the salt is at least 50 mM, at least about 100 mM, at least about 150 mM, at least about 200 mM, at least about 250 mM, at least about 260 mM, at least about 270 mM, at least about 280 mM, at least about 290 mM, at least about 300 mM, at least about 310 mM, at least about 320 mM, at least about 330 mM, at least about 340 mM, at least about 350 mM, at least about 360 mM, at least about 370 mM, at least about 380 mM, at least about 390 mM, at least about 400 mM, at least about 450 mM, at least about 500 mM, at least about 550 mM, or at least about 600 mM.

In some aspects, the pH of the pH gradient is between about pH 3 and about pH 11. In some aspects, the mixture is in a buffer. In some aspects, the buffer comprises MES, phosphate buffer, Tris, or any combination thereof.

In some aspects, the method further comprises measuring a post-translational modification. In some aspects, the post-translational modification comprises N-glutamine pyroglutamation, C-terminal lysine truncation, C-terminal proline amidation, glycation, sialylation, deamidation, aspartic acid isomerization, generic truncation, or any combination thereof.

In some aspects, the protein comprises a fusion protein or an antibody or antigen binding portion thereof. In some aspects, the antibody or antigen binding portion thereof binds an antigen selected from PD-1, PD-L1, CTLA-4, LAG-3, TIGIT, GITR, CXCR4, CD73, HER2, VEGF, CD20, CD40, CD11a, tissue factor (TF), MICA/B PSCA, IL-8, EGFR, HER3, HER4, and any combination thereof.

In some aspects, the fusion protein comprises an immunoglobulin component and a growth factor. In some aspects, the fusion protein comprises an Fc-fusion protein. In some aspects, the fusion protein comprises an Fc fused to a CTLA-4.In some aspects, the fusion protein comprises abatacept or belatacept.

In some aspects, the fusion protein comprises an Fc fused to an interleukin.

In some aspects, the species of the fusion protein or antibody is an acidic species, a basic species or a main species. In some aspects, the fusion protein or antibody is partially purified by protein A affinity chromatography.

In some aspects, the species is enriched by a counter current purification system. In some aspects, the counter current purification system is a Multicolumn Counter Current Solvent Gradient Purification (MCSGP) system.

Some aspects of the present disclosure are directed to a species of a protein prepared by a method disclosed herein. In some aspects, the species of the protein is a charge variant.

Some aspects of the present disclosure are directed to a method of treating a disease or condition in a subject in need thereof comprising administering the species of a protein disclosed herein.

### BRIEF DESCRIPTION OF THE DRAWINGS/FIGURES

FIGS. 1A, 1B, and 1C provide a schematic of the enrichment method described herein using the MCSGP twin-column continuous chromatography. The load material is represented by a mixture of three species (red (“R”), green (“G”), and blue (“B”)), where the green species is the species of interest. Enrichment of the green species is achieved through a three-stage operation including enrichment of the green species, depletion of the red and blue species, and elution of the green species. Specifically, FIG. 1A shows the enrichment stage, in which the mixture is loaded onto the first column and, after discarding the species that elutes from the column first (blue), the species for enrichment (green) is recycled to the second column. After all of the green component is recycled, the later eluting species (red) is discarded from the first column via a strip step. Additional load (mixture) is injected to the column 2 and the same separation from column 1 (discarding blue, recycling green, and discarding red) is then conducted on column 2, and enrichment of green occurs as the number of cycles increases. FIG. 1B shows the depletion stage, where the species of interest (green) stays in the column and the unwanted species (blue and red) are removed. Depletion is followed by the elution stage to recover the green species from the system. FIG. 1C shows a simulation of the UV trace for the fore mentioned operation as a whole. In the left panel, the load material contains the same abundance of the three species, blue, green and red. The middle panel shows the resulting product following 10 cycles of enrichment of the green species. Finally, the right panel shows that after 1 cycle of depletion, there is virtually none of the blue and red species left in the system, while the green species remains unaltered. This product is eluted during the final stage of processing and collected for subsequent analysis. The duration of each stage for one of the molecules discussed in this paper was about 45 minutes per cycle. Given that there were 10 cycles of enrichment, 2 cycles of depletion, and 1 cycle of elution, this totaled about 10 hours of processing time.

FIGS. 2A, 2B, 2C, and 2D show the charge variant separation of the mAb1 antibody using one of the following chromatography systems: (i) high performance liquid chromatography (HPLC), (ii) fast protein liquid chromatography (FPLC), and (iii) continuous chromatography described herein (CUBE). FIG. 2A shows the charge variant separation using the HPLC system. The fraction before the arrow is defined as the acidic fraction. FIG. 2B shows the charge variant separation using the FPLC system. The fraction before the arrow is defined as the acidic fraction. FIG. 2C shows the acidic fraction enrichment using CUBE. Comparison of continuous chromatography profiles between cycle 2 (black; 1) and cycle 10 (red; 2) from column 1 (left) and column 2 (right) is provided. FIG. 2D provides comparison of the processing time (black (1) bars) and acidic fraction purity (red (2) bars) with separation, or enrichment, methods using HPLC, FPLC, and continuous chromatography methods. The time was calculated for generating 10 mg of acidic fraction from a load material with 17% acidic variant. Purity was determined with iCIEF method.

FIG. 3 provides image capillary electrophoresis profiles of charge variants of the mAb1 antibody isolated using continuous chromatography. Panel (1) shows the load material before isolation or enrichment. Both the acidic and basic regions are identified. Panel (2) shows the enrichment of acidic region 1 shown in Panel (1). Panel (3) shows the enrichment of acidic region 2 shown in Panel (1). Panel (4) shows the enrichment of basic region 1 shown in Panel (1). And, Panel (5) shows the enrichment of basic region 2 shown in Panel (1). Y-axis is normalized based on the peak maximum.

FIG. 4 provides image capillary electrophoresis profiles of charge variants of the mAb2 antibody isolated using continuous chromatography. The top panel (a) is for the load material before fractionation or enrichment. The second panel (b) is the load material after enrichment. The arrows indicate the acidic species present in the samples. Y-axis is normalized based on the peak maximum.

FIG. 5A provides the intact mass of deglycosylated samples from mAb3 (from top to bottom panels: standard reference material, unenriched material, enriched acidic species, and enriched basic species). The peak around 144673 m/z is assigned to deglycosylated species. Peaks around 144835 m/z and 144999 m/z are assigned to species with one and two glycations, respectively. The peak around 144558 m/z in the basic sample, which is barely visible in the unenriched material, is assigned to C-terminal proline amidation species.

FIG. 5B shows the presence of truncated species in the deglycosylated samples from FIG. 4A (from top to bottom panels are standard reference material, load material, enriched acidic species, and enriched basic species). The peak around 121385 m/z is assigned as species missing one light chain. The peak around 131538 m/z is assigned to heavy chain truncated species with one heavy chain missing residues after 329.

FIG. 6 provides image capillary electrophoresis profiles of charge variants of the mAb3 antibody. Acidic species of mAb3 were fractioned (with FPLC) or enriched (with continuous chromatography) using Mono S CEX or Mono Q AEX with salt gradient or pH gradient. Panel (1) shows the load material before fractionation or enrichment. Panel (2) shows the fractioned acidic species using Mono S with salt gradient with FPLC. Panel (3) shows the enriched acidic species using Mono S with salt gradient with continuous chromatography. Panel (4) shows the fractioned acidic species using Mono Q with pH gradient with FPLC. Panel (5) shows the enriched acidic species using Mono Q with pH gradient with continuous mode. Y-axis is normalized based on the peak maximum.

FIGS. 7A, 7B, 7C, 7D, and 7E provide the peptide mapping mass spectrometry results for the mAb3 antibody samples isolated using cation exchange chromatography (CEX) (Mono S) and anion exchange chromatography (AEX) (Mono Q). Samples labeled as “Fractioned” were prepared using traditional FPLC methodology, and samples labeled as “Enriched” were prepared using continuous chromatography methodology. FIG. 7A shows the sialylation of samples isolated using Mono S with salt gradient (top panel) or using Mono Q with pH gradient (bottom panel). The bars represent different sialylated glycoforms that were detected: GIFS (white), G2FS (light gray), and G2FS2 (dark gray). G1FS is the mono-sialylated glycoform of the G1F glycan. G2FS and G2FS2 are the mono-and di-sialylated glycoforms of the G2F glycan, respectively. FIG. 7B shows the deamidation of samples isolated using Mono S with salt gradient (top panel) or using Mono Q with pH gradient (bottom panel). The bars represent different amino acid residues: N84 (white), N325 (light gray), N384 (gray), and N389 (dark gray). FIG. 7C shows the glycation of samples isolated using Mono S with salt gradient (top panel) or using Mono Q with pH gradient (bottom panel). The bars represent the different peptides: peptide 1 (white) and peptide 2 (gray). FIG. 7D shows the percentage of N-terminal glutamine detected in samples isolated using Mono S with salt gradient (top panel) or using Mono Q with pH gradient (bottom panel). FIG. 7E shows the percentage of C-terminal proline amidation observed in samples isolated using Mono S with salt gradient (top panel) or using Mono Q with pH gradient (bottom panel).

### DETAILED DESCRIPTION

Protein purification and isolation on a large scale can be costly and time consuming. Traditional methods of separating species of a particular protein rely on HPLC and/or FPLC. However, getting substantial yields, such as great than 10 mg of a desired protein species, can take several weeks or longer. Some aspects of the present disclosure are directed to methods of isolating a species of a protein from a mixture comprising the species and one or more impurities, comprising contacting the mixture with two or more chromatography columns in a continuous operation mode. Some aspects of the present disclosure are directed to methods of increasing purity and/or yield of a species of a protein from a mixture comprising the species and one or more impurities, comprising contacting the mixture with two or more chromatography columns in a continuous operation mode.

Some aspects of the present disclosure are directed to methods for enriching a species of a protein for analytical characterization, comprising: (a) separating the species of the protein from a mixture comprising the species and one or more impurities, comprising contacting the mixture with two or more chromatography columns in a continuous operation mode in a chromatographic separation system; and (b) subjecting the species from (a) to analytical characterization.

Some aspects of the present disclosure are directed to methods for conducting analytical characterization of a species of a protein, comprising: (a) separating the species of the protein from a mixture comprising the species and one or more impurities, comprising contacting the mixture with two or more chromatography columns in a continuous operation mode in a chromatographic separation system; and (b) conducting analytical characterization of the species from (a).

In some aspects, the species of the protein is a charge variant. In some aspects, the two or more chromatography columns comprise at least two ion exchange columns. In some aspects, the two or more chromatography columns comprise a pH gradient. In some aspects, the two or more chromatography columns comprise a salt gradient. In some aspects, the two or more chromatography columns comprise a pH gradient and a salt gradient.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

It is understood that wherever aspects are described herein with the language “comprising,” otherwise analogous aspects described in terms of “consisting of” and/or “consisting essentially of” are also provided.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. The headings provided herein are not limitations of the various aspects of the disclosure, which can be had by reference to the specification as a whole. Accordingly, the terms defined are more fully defined by reference to the specification in its entirety.

Abbreviations used herein are defined throughout the present disclosure. Various aspects of the disclosure are described in further detail in the following subsections.

The terms “purifying,” “separating,” or “isolating,” as used interchangeably herein, refer to increasing the degree of purity of a protein of interest from a composition or sample comprising the protein of interest and one or more impurities. Typically, the degree of purity of the protein of interest is increased by removing (completely or partially) at least one impurity from the composition. In some aspects, the protein of interest is a first charge variant of a protein, e.g., a charge variant of an antibody, and the one or more impurities comprises a second charge variant of the same protein.

The term “chromatography,” as used herein, refers to a dynamic separation technique, which separates a target molecule such as a target protein (e.g., a charge variant of a protein, e.g., an antibody) from other molecules in the mixture (e.g., other charge variants) and allows it to be isolated. Typically, in a chromatography method, a liquid mobile phase transports a sample containing the target molecule of interest across or through a stationary phase (normally solid) medium. Differences in partition or affinity to the stationary phase causes the temporary binding of selected molecules to the stationary phase while the mobile phase carries different molecules out at different times.

The term “continuous operation mode” or “continuous chromatography” refers to chromatography process wherein a sample is passed through at least two chromatography columns in tandem (i.e., the elution from a first column is loaded directly onto a second column). In some aspects, a sample is loaded onto a first column, the elution from the first column is applied directly to a second column, and the elution from the second column is collected. In some aspects, a sample is loaded onto a first column, the elution from the first column is loaded directly onto a second column, and the elution from the second column is loaded back onto the first column, and the process is repeated at least one time, at least two times, at least three times, at least four times, or at least five times before the elution from the second column is collected.

The term “ion exchange chromatography,” as used herein, refers to a mode of chromatography where a target molecule, such as a protein (e.g., a charge variant of a protein) to be separated is isolated based on polar interactions with charged molecules (e.g., positively or negatively charged molecules) immobilized on the chromatography resin. Elution from an ion exchange chromatography column can be achieved using a salt gradient or changing the pH.

An “anion exchange chromatography” or “AEX” refers to an ion exchange chromatography comprising a positively charged ion exchange resin with an affinity for molecules having net negative surface charges. A salt gradient can be applied to the column to separate a protein of interest from other bound proteins, and proteins will be eluted in an order depending on their net surface charge.

A “cation exchange chromatography” or “CEX refers to an ion exchange chromatography comprising a negatively charged ion exchange resin with an affinity for molecules having net positive surface charges. A salt gradient can be applied to the column to separate a protein of interest from other bound proteins, and proteins will be eluted in an order depending on their net surface charge.

The term “affinity chromatography,” as used herein, refers to a mode of chromatography where a target molecule, such as a protein molecule (e.g., a charge variant of a protein), to be separated is isolated by its “lock-and-key” interaction with a molecule (e.g., a Protein A based ligand) immobilized on the chromatography resin. This specific interaction allows the target molecule to bind the molecule immobilized on the resin while the undesirable molecules flow through. Changing the temperature, pH, or ionic strength of the mobile phase then releases the target molecule in high purity. In various embodiments described herein, affinity chromatography involves the addition of a sample containing a target molecule (e.g., an immunoglobulin or another Fc-containing protein) to a solid support which carries on it a ligand based on the C domain of Protein A (referred to as Protein A affinity chromatography media or resin). Other ligands used for affinity chromatography can include, e.g., Protein G from Steptococci which binds to the Fc region of a immunoglobulin.

The term “high-performance liquid chromatography,” or “HPLC,” or “high-pressure liquid chromatography,” as used herein, refers to a chromatographic system that relies on pumps to pass a pressurized liquid and a sample mixture through a column filled with adsorbent, leading to the separation of the sample components. The components of the sample mixture are separated from each other due to their different degrees of interaction with the adsorbent particles.

The term “capillary isoelectric focusing gel electrophoresis” or “cIEF gel electrophoresis,” as used herein, refers to a high-resolution analytical technique that allows the separation of protein/peptide mixtures, protein glycoforms, and other charge variants, based on their isoelectric point (pI).

The term “imaged capillary isoelectric focusing” or “iCIEF,” as used herein, refers to an analytical technique that separates ampholytic components of biomolecules in an electric field according to their isoelectric points.

The term “contacting” as used herein, refers to applying a solution, e.g., a mixture comprising a protein product and a contaminant, as described herein, to a chromatography matrix. In some embodiments, the term “contacting” is synonymous with “loading” a solution onto a chromatography column. A “column packing” or a “chromatography matrix” as used herein refers to the adsorbent solid material contained within a chromatography column. In some aspects, the column packing comprises Super Q. In some aspects, the column packing comprises GigaCap. In some aspects, the column packing comprises FRACTOGEL® SO₃⁻.

The term “is applied to,” when used in the context of a gradient being applied to a chromatography matrix, broadly means that a gradient is formed, directly or indirectly, within and/or around a chromatography matrix. In some embodiments, the chromatography matrix is present in a column, and the gradient is formed within the column. In some embodiments, a gradient that is applied to a chromatography matrix is formed internally within a column, as opposed to a gradient which is formed externally and then added to a column. In certain embodiments, a gradient that is applied to the chromatography matrix forms within a column as a result of more than one buffer being added to the chromatography matrix. In other embodiments, a gradient that is applied to the chromatography matrix is formed externally and then added to the column.

The terms “culture”, “cell culture” and “eukaryotic cell culture” as used herein refer to a cell population, either surface-attached or in suspension that is maintained or grown in a medium (see definition of “medium” below) under conditions suitable to survival and/or growth of the cell population. As will be clear to those of ordinary skill in the art, these terms as used herein can refer to the combination comprising the cell population and the medium in which the population is suspended.

As used herein, the terms “expression” or “expresses” are used to refer to transcription and translation occurring within a cell. The level of expression of a product gene in a host cell can be determined on the basis of either the amount of corresponding mRNA that is present in the cell or the amount of the protein encoded by the product gene that is produced by the cell, or both.

The term “antibody” refers, in some aspects, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The term “antibody” can include a bispecific antibody or a multispecific antibody.

An “IgG antibody”, e.g., a human IgG1, IgG2, IgG3 and IgG4 antibody, as used herein has, in some aspects, the structure of a naturally-occurring IgG antibody, i.e., it has the same number of heavy and light chains and disulfide bonds as a naturally-occurring IgG antibody of the same subclass. For example, an IgG1, IgG2, IgG3 or IgG4 antibody may consist of two heavy chains (HCs) and two light chains (LCs), wherein the two HCs and LCs are linked by the same number and location of disulfide bridges that occur in naturally-occurring IgG1, IgG2, IgG3 and IgG4 antibodies, respectively (unless the antibody has been mutated to modify the disulfide bridges).

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment (fragment from papain cleavage) or a similar monovalent fragment consisting of the VL, VH, LC and CHI domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” refers to a molecule composed of monomers (amino acids) linearly linked by amide bonds (also known as peptide bonds). The terms “polypeptide” or “protein” or “product” or “product protein” or “amino acid residue sequence” are used interchangeably. The term “polypeptide” refers to any chain or chains of two or more amino acids, and does not refer to a specific length of the product. As used herein the term “protein” is intended to encompass a molecule comprised of one or more polypeptides, which can in some instances be associated by bonds other than amide bonds. On the other hand, a protein can also be a single polypeptide chain. In this latter instance the single polypeptide chain can in some instances comprise two or more polypeptide subunits fused together to form a protein. The terms “polypeptide” and “protein” also refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology.

The terms “polynucleotide” or “nucleotide” as used herein are intended to encompass a singular nucleic acid as well as plural nucleic acids, and refers to an isolated nucleic acid molecule or construct, e.g., messenger RNA (mRNA), complementary DNA (cDNA), or plasmid DNA (pDNA). The term “nucleic acid” refers to any one or more nucleic acid segments, e.g., DNA, cDNA, or RNA fragments, present in a polynucleotide. When applied to a nucleic acid or polynucleotide, the term “isolated” refers to a nucleic acid molecule, DNA or RNA, which has been removed from its native environment, for example, a recombinant polynucleotide encoding an antigen binding protein contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

The term “impurity” or “impurities,” as used herein, refers to one or more molecule, e.g., polypeptide, nucleic acid molecule, small molecule, or any combination thereof, present in a mixture with a target molecule, e.g., a target species of a polypeptide, e.g., a target charge variant of a polypeptide. In some aspects, the impurity is a different polypeptide, e.g., a polypeptide that has a different structure, sequence, or function than a target polypeptide. In some aspects, the impurity is a different species of a target polypeptide, e.g., a charge variant or a HMW species.

The term “purity,” as used herein, refers to the degree to which a composition, e.g., a solution comprising a target polypeptide, comprises one or more impurities. For example, a solution comprising a target polypeptide wherein 98% of the target polypeptide in the solution is charge variant A, and 2% of the target polypeptide comprises one or more other charge variants than charge variant A, has a purity of 98%.

### II. Methods of the Disclosure

Some aspects of the present disclosure are directed to methods of isolating a species of a protein from a mixture comprising the species and one or more impurities, comprising contacting the mixture with two or more chromatography columns in a continuous operation mode. Some aspects of the present disclosure are directed to methods of increasing purity and/or yield of a species of a protein from a mixture comprising the species and one or more impurities, comprising contacting the mixture with two or more chromatography columns in a continuous operation mode. In some aspects, the chromatography comprises a counter current purification system. In some aspects, the counter current purification system is a Multicolumn Counter Current Solvent Gradient Purification (MCSGP) system.

The term “species” or “variants” of a protein refers to difference forms which are encoded by the same nucleotide sequence, but differ in protein chain length, protein mass and/or post-translational modification, including but not limited to, species varying in the degree of glycosylation, monomers, oligomers or multimers (also referred to as high molecule weight (HMW) species, truncated forms, charge forms, etc. For example, monoclonal antibodies (mAbs) are heterogeneous in their biochemical and biophysical properties due to multiple posttranslational modification and degradation events. Charge heterogeneity of mAbs can be affected by these modifications resulting in alteration of the net charge or the local charge distribution. Charge variants of mAbs are identified as acidic species, basic species and main species. The term “main species”, “main peak” or “main variant” of mAb as used herein refers to the mAb that is eluted as the major peak with neutral Isoelectric Point (pI). The term “acidic species” or “acidic variant” of mAbs used herein refers to the variants with lower pI than the main species. The term “basic species” or “basic variant” of mAb as used herein refers to the variants with higher pI than the main species. C-terminal Lysine residues of the mAb can result in additional positive charges and increasing the basic species of the mAb. The non-efficient cleavage of C-terminal lysine residues by endogenous carboxypeptidases during the antibody production is one of the major reasons to result in a mAb with zero, one or two C-terminal lysine (Zhang et al., 2015).

In some aspects, the methods disclosed herein result in increased purity of the species, as compared to conventional methods. In some aspects, the methods disclosed herein result in increased purity of the species, as compared to HPLC or FPLC methods. In some aspects, the purity of the sample is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold, as compared to HPLC or FPLC methods.

In some aspects, the methods disclosed herein reduce the total time necessary to obtain a sufficient amount of the species, as compared to conventional methods. In some aspects, the methods disclosed herein reduce the total time necessary to obtain a sufficient amount of the species, as compared to HPLC or FPLC methods. In some aspects, the sufficient amount of the species is at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 20 mg, at least about 25 mg, at least about 30 mg, at least about 35 mg, at least about 40 mg, at least about 45 mg, at least about 50 mg, at least about 75 mg, or at least about 100 mg of the species. In some aspects, the sufficient amount of the species is at least about 10 mg. In some aspects, the time necessary to obtain a sufficient amount of the species is reduced by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold, relative to conventional methods (e.g., HPLC or FPLC). In some aspects, the time necessary to obtain a sufficient amount of the species is less than about 90%, less than about 80%, less than about 75%, less than about 70%, less than about 60%, less than about 50%, less than about 40%, less than about 30%, less than about 25%, less than about 20%, less than about 15%, less than about 10%, less than about 5%, less than about 4%, less than about 3%, less than about 2%, or less than about 1% that of the time necessary to obtain the same or comparable amount of the species using conventional methods, e.g., HPLC or FPLC. In some aspects, the methods disclosed herein yield at least about 10 mg of a target species in less than about 7 days, less than about 6 days, less than about 5 days, less than about 4 days, less than about 3 days, less than about 48 hours, less than about 36 hours, less than about 30 hours, or less than about 24 hours. In some aspects, the methods disclosed herein yield at least about 10 mg of a target species in less than about 48 hours. In some aspects, the methods disclosed herein yield at least about 10 mg of a target species in less than about 36 hours. In some aspects, the methods disclosed herein yield at least about 10 mg of a target species in less than about 30 hours. In some aspects, the methods disclosed herein yield at least about 10 mg of a target species in less than about 24 hours.

In some aspects, the methods disclosed herein have increased productivity (measured by normalizing the yield (e.g., grams of species) to the duration) relative to conventional methods, e.g., HPLC or FPLC. In some aspects the productivity is increased by at least about at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, or at least about 30-fold, relative to conventional methods (e.g., HPLC or FPLC).

In some aspects, the method further comprises subjecting the isolated species to one or more analytical characterization. As such, some aspects of the present disclosure are directed to methods for enriching a species of a protein for analytical characterization, comprising: (a) separating the species of the protein from a mixture comprising the species and one or more impurities, comprising contacting the mixture with two or more chromatography columns in a continuous operation mode in a chromatographic separation system; and (b) subjecting the species from (a) to analytical characterization. Some aspects of the present disclosure are directed to methods for conducting analytical characterization of a species of a protein, comprising: (a) separating the species of the protein from a mixture comprising the species and one or more impurities, comprising contacting the mixture with two or more chromatography columns in a continuous operation mode in a chromatographic separation system; and (b) conducting analytical characterization of the species from (a).

In some aspects, the analytical characterization comprises a HPLC system, capillary isoelectric focusing (cIEF) gel electrophoresis, imaged capillary isoelectric focusing (iCIEF), cation exchange chromatography (CEX), anion exchange chromatography (AEX), MFI, SEC-MALS, SEC, mass spectrometry, or any combination thereof. In some aspects, the analytical characterization comprises subjecting the species, e.g., the charge variant of a protein (e.g., antibody), to an HPLC system. In some aspects, the analytical characterization comprises subjecting the species, e.g., the charge variant of a protein (e.g., antibody), to a capillary isoelectric focusing (cIEF) gel electrophoresis. In some aspects, the analytical characterization comprises subjecting the species, e.g., the charge variant of a protein (e.g., antibody), to imaged capillary isoelectric focusing (iCIEF). In some aspects, the analytical characterization comprises subjecting the species, e.g., the charge variant of a protein (e.g., antibody), to cation exchange chromatography (CEX). In some aspects, the analytical characterization comprises subjecting the species, e.g., the charge variant of a protein (e.g., antibody), to anion exchange chromatography (AEX).

### A. Column Chromatography

Some aspects of the present disclosure comprise contacting a mixture comprising a species of a protein and one or more impurities to two or more chromatography columns in a continuous operation mode. In some aspects, the two or more chromatography columns enrich the species, e.g., the charge variant. In some aspects, the method comprises loading the mixture comprising a species of a protein and one or more impurities on to a first chromatography column. The first chromatography column can comprise any chromatography matrix. In some aspects, the chromatography matrix of the first column is an AEX matrix. In some aspects, the chromatography matrix of the first column is an CEX matrix. In some aspects, the chromatography matrix of the first column is a mixed-mode chromatography matrix. In some aspects, the chromatography matrix of the first column is an affinity chromatography matrix. In some aspects, the chromatography matrix of the first column is a size exclusion matrix.

In some aspects, the loaded mixture passes through the first chromatography column and is separated into (i) an enrichment species comprising the species and (ii) one or more discard species comprising one or more impurities. This step is referred to herein as “enrichment stage I.” During enrichment stage I, the discard species is eluted from the column and discarded. The enrichment species exiting the column is then loaded onto a second column. In some aspects, the second column is positioned such that the enrichment species is eluted from the first column directly onto the second column. In some aspects, the enrichment species is collected from the first column and applied to the second column. In some aspects, a second discard species is eluted from the first column and discarded after the enrichment species has passed through the column, i.e., a second discard species that travels more slowly across the column than the enrichment species is eluted and discarded from the first column.

In some aspects, the enrichment species is applied to a second column. The enrichment species travels through the second column and is further separated into (i) an enrichment species comprising the species and (ii) one or more additional discard species comprising one or more additional impurities. This step is referred to herein as “enrichment stage II.” During enrichment stage II, the additional discard species is eluted from the column and discarded. In some aspects, the additional discard species elutes from the second column before the enrichment species. In some aspects, the additional discard species elutes from the second column after the enrichment species. In some aspects, an additional discard species elutes from the second column before the enrichment species, and an additional discard species elutes from the second column after the enrichment species.

Once the enrichment species passes through the second column, the enrichment species is loaded onto the first column. In some aspects, additional mixture (comprising the species and one or more impurities) is added to the first column at the same time as the enrichment species. In some aspects, the enrichment species is combined with the additional mixture prior to loading onto the first column. In some aspects, the enrichment species is loaded onto the first column, and then the additional mixture is loaded onto the same first column. In some aspects, the additional mixture is loaded onto the first column, and then the enrichment species is loaded onto the same first column. In some aspects, the additional mixture is added after the enrichment species is added to the first chromatography column, and before the enrichment species passes through the first chromatography column. In some aspects, the first column is re-equilibrated prior to loading. In some aspects, the second column is re-equilibrated prior to loading.

In some aspects, enrichment stages I and II are repeated at least twice, at least three times, at least four times, at least five times, at least six times, at least seven times, at least eight times, at least nine times, or at least ten times. In some aspects, enrichment stages I and II are repeated until all starting mixture has been applied to the first column.

In some aspects, the method further comprises a “depletion stage.” The depletion stage follows the enrichment stage, i.e., after enrichment stages I and II are repeated n times, the enrichment species proceeds to the depletion stage. In some aspects, n is at least 2, at least 3, at least 4, at least 5, at least 6, at least 7, at least 8, at least 9, or at least 10. In some aspects, the depletion stage comprises contacting the enrichment species with the first chromatography column in the absence of an additional mixture. The enrichment species then passes through the first chromatography column, separating the charge species from one or more remaining impurities. In some aspects, a first remaining impurity exits the first column before the enrichment species, and the first remaining impurity is discarded. In some aspects, the enrichment species exits the first column and is applied to the second column. The enrichment species then passes through the second chromatography column, separating the enrichment species from one or more remaining impurities. Following the depletion stage, the species is eluted from the second column.

In some aspects, the eluted species has a purity of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100%. In some aspects, the eluted species has a purity of at least about 90%. In some aspects, the eluted species has a purity of at least about 95%. In some aspects, the eluted species has a purity of at least about 96%. In some aspects, the eluted species has a purity of at least about 97%. In some aspects, the eluted species has a purity of at least about 98%. In some aspects, the eluted species has a purity of at least about 99%.

In some aspects, the species is eluted at a concentration that is at least about 1.5, at least about 1.6, at least about 1.7, at least about 1.8, at least about 1.9, at least about 2.0, at least about 2.5, at least about 3.0, at least about 3.5, at least about 4.0, at least about 4.5, at least about 5.0, at least about 5.5, at least about 6.0, at least about 6.5, at least about 7.0, at least about 7.5, at least about 8.0, at least about 8.5, at least about 9.0, at least about 9.5, or at least about 10.0 fold higher than the concentration of the species in the mixture.

In some aspects, the method further comprises measuring a post-translational modification on the species of the protein. In some aspects, the modification comprises N-glutamine pyroglutamation, C-terminal lysine truncation, C-terminal proline amidation, glycation, sialylation, deamidation, aspartic acid isomerization, generic truncation, or any combination thereof.

In some aspects, one or more of the chromatography columns comprise a salt gradient. In some aspects, one or more of the chromatography columns comprise a pH gradient. In some aspects, one or more of the chromatography columns comprise a salt gradient and a pH gradient. In some aspects, the salt gradient comprises a sodium chloride (NaCl gradient). In some aspects, the salt gradient comprises a gradient from no salt, e.g., no NaCl, to a high salt concentration.

In some aspects, the salt (e.g., NaCl) concentration is from 0 mM to at least about 500 mM, 0 mM to at least about 450 mM, 0 mM to at least about 400 mM, 0 mM to at least about 350 mM, 0 mM to at least about 300 mM, 0 mM to at least about 290 mM, 0 mM to at least about 280 mM, 0 mM to at least about 270 mM, 0 mM to at least about 260 mM, 0 mM to at least about 250 mM, about 50 mM to at least about 500 mM, about 50 mM to at least about 450 mM, about 50 mM to at least about 400 mM, about 50 mM to at least about 350 mM, about 50 mM to at least about 300 mM, about 50 mM to at least about 290 mM, about 50 mM to at least about 280 mM, about 50 mM to at least about 270 mM, about 50 mM to at least about 260 mM, about 50 mM to at least about 250 mM, about 100 mM to at least about 500 mM, about 100 mM to at least about 450 mM, about 100 mM to at least about 400 mM, about 100 mM to at least about 350 mM, about 100 mM to at least about 300 mM, about 100 mM to at least about 290 mM, about 100 mM to at least about 280 mM, about 100 mM to at least about 270 mM, about 100 mM to at least about 260 mM, about 100 mM to at least about 250 mM, about 150 mM to at least about 350 mM, about 1200 mM to at least about 300 mM, or about 225 mM to at least about 375 mM salt (e.g., NaCl).

In some aspects, the concentration of the salt (e.g., NaCl) is at least 50 mM, at least about 100 mM, at least about 150 mM, at least about 200 mM, at least about 250 mM, at least about 260 mM, at least about 270 mM, at least about 280 mM, at least about 290 mM, at least about 300 mM, at least about 310 mM, at least about 320 mM, at least about 330 mM, at least about 340 mM, at least about 350 mM, at least about 360 mM, at least about 370 mM, at least about 380 mM, at least about 390 mM, at least about 400 mM, at least about 450 mM, at least about 500 mM, at least about 550 mM, or at least about 600 mM salt (e.g., NaCl).

In some aspects, the salt gradient is a linear gradient. In some aspects, the salt gradient is a step gradient.

In some aspects, the salt gradient mobile phase further comprises a buffer. In some aspects, the salt gradient mobile phase comprises MES. In some aspects, the salt gradient mobile phase comprises at least about 10 mM MES, at least about 15 mM MES, at least about 20 mM MES, at least about 25 mM MES, or at least about 30 mM MES. In some aspects, the salt gradient mobile phase comprises at least about 20 mM MES. In some aspects, the pH of the salt gradient mobile phase is at least about 5.5, at least about 5.6, at least about 5.7, at least about 5.8, at least about 5.9, at least about 6.0, at least about 6.1, at least about 6.2, at least about 6.3, at least about 6.4, or at least about 6.5. In some aspects, the salt gradient mobile phase comprises 20 mM MES with and without 250 mM sodium chloride at pH 6.0. In some aspects, the salt gradient mobile phase comprises 20 mM MES, pH 5.8 with and without 400 mM sodium chloride.

In some aspects, one or more of the chromatography columns comprise a pH gradient. In some aspects, the pH of the pH gradient mobile phase is between about pH 3 and about pH 11, about pH 3 and about pH 10, about pH 3 and about pH 9, about pH 3 and about pH 8, about pH 3 and about pH 7, about pH 4 and about pH 11, about pH 5 and about pH 11, about pH 6 and about pH 11, or about pH 7 and about pH 11. In some aspects, the pH of the pH gradient mobile phase is between about pH 3 and about pH 11.

In some aspects, the pH gradient mobile phase further comprises a buffer. In some aspects, the buffer comprises MES, phosphate buffer, Tris, bis-Tris, 1,3 diaminopropane, diethanolamine, piperazine, imidazole, acetic acid, malonic acid, formic acid, MOPSO, HEPES, BICINE, CHES, CAPS, or any combination thereof.

In some aspects, the buffer comprises at least about 10 mM MES, at least about 15 mM MES, at least about 20 mM MES, at least about 25 mM MES, or at least about 30 mM MES. In some aspects, the buffer comprises at least about 20 mM MES.

In some aspects, the buffer comprises at least about 1 mM Tris, at least about 2 mM Tris, at least about 3 mM Tris, at least about 4 mM Tris, at least about 5 mM Tris, at least about 6 mM Tris, at least about 7 mM Tris, at least about 8 mM Tris, at least about 9 mM Tris, and at least about 10 mM Tris. In some aspects, the buffer comprises at least about 5 mM Tris.

In some aspects, the buffer comprises at least about 1 mM bis-Tris, at least about 2 mM bis-Tris, at least about 3 mM bis-Tris, at least about 4 mM bis-Tris, at least about 5 mM bis-Tris, at least about 6 mM bis-Tris, at least about 7 mM bis-Tris, at least about 8 mM bis-Tris, at least about 9 mM bis-Tris, and at least about 10 mM bis-Tris. In some aspects, the buffer comprises at least about 5 mM bis-Tris.

In some aspects, the buffer comprises at least about 1 mM 1,3 diaminopropane, at least about 2 mM 1,3 diaminopropane, at least about 3 mM 1,3 diaminopropane, at least about 4 mM 1,3 diaminopropane, at least about 5 mM 1,3 diaminopropane, at least about 6 mM 1,3 diaminopropane, at least about 7 mM 1,3 diaminopropane, at least about 8 mM 1,3 diaminopropane, at least about 9 mM 1,3 diaminopropane, and at least about 10 mM 1,3 diaminopropane. In some aspects, the buffer comprises at least about 5 mM 1,3 diaminopropane.

In some aspects, the buffer comprises at least about 1 mM diethanolamine, at least about 2 mM diethanolamine, at least about 3 mM diethanolamine, at least about 4 mM diethanolamine, at least about 5 mM diethanolamine, at least about 6 mM diethanolamine, at least about 7 mM diethanolamine, at least about 8 mM diethanolamine, at least about 9 mM diethanolamine, and at least about 10 mM diethanolamine. In some aspects, the buffer comprises at least about 5 mM diethanolamine.

In some aspects, the buffer comprises at least about 1 mM piperazine, at least about 2 mM piperazine, at least about 3 mM piperazine, at least about 4 mM piperazine, at least about 5 mM piperazine, at least about 6 mM piperazine, at least about 7 mM piperazine, at least about 8 mM piperazine, at least about 9 mM piperazine, and at least about 10 mM piperazine. In some aspects, the buffer comprises at least about 5 mM piperazine.

In some aspects, the buffer comprises at least about 1 mM imidazole, at least about 2 mM imidazole, at least about 3 mM imidazole, at least about 4 mM imidazole, at least about 5 mM imidazole, at least about 6 mM imidazole, at least about 7 mM imidazole, at least about 8 mM imidazole, at least about 9 mM imidazole, and at least about 10 mM imidazole. In some aspects, the buffer comprises at least about 5 mM imidazole.

In some aspects, the buffer comprises at least about 1 mM acetic acid, at least about 2 mM acetic acid, at least about 3 mM acetic acid, at least about 4 mM acetic acid, at least about 5 mM acetic acid, at least about 6 mM acetic acid, at least about 7 mM acetic acid, at least about 8 mM acetic acid, at least about 9 mM acetic acid, and at least about 10 mM acetic acid. In some aspects, the buffer comprises at least about 5 mM acetic acid.

In some aspects, the buffer comprises at least about 1 mM malonic acid, at least about 2 mM malonic acid, at least about 3 mM malonic acid, at least about 4 mM malonic acid, at least about 5 mM malonic acid, at least about 6 mM malonic acid, at least about 7 mM malonic acid, at least about 8 mM malonic acid, at least about 9 mM malonic acid, and at least about 10 mM malonic acid. In some aspects, the buffer comprises at least about 5 mM malonic acid.

In some aspects, the buffer comprises at least about 1 mM formic acid, at least about 2 mM formic acid, at least about 3 mM formic acid, at least about 4 mM formic acid, at least about 5 mM formic acid, at least about 6 mM formic acid, at least about 7 mM formic acid, at least about 8 mM formic acid, at least about 9 mM formic acid, and at least about 10 mM formic acid. In some aspects, the buffer comprises at least about 5 mM formic acid.

In some aspects, the buffer comprises at least about 1 mM MOPSO, at least about 2 mM MOPSO, at least about 3 mM MOPSO, at least about 4 mM MOPSO, at least about 5 mM MOPSO, at least about 6 mM MOPSO, at least about 7 mM MOPSO, at least about 8 mM MOPSO, at least about 9 mM MOPSO, and at least about 10 mM MOPSO. In some aspects, the buffer comprises at least about 5 mM MOPSO.

In some aspects, the buffer comprises at least about 1 mM HEPES, at least about 2 mM HEPES, at least about 3 mM HEPES, at least about 4 mM HEPES, at least about 5 mM HEPES, at least about 6 mM HEPES, at least about 7 mM HEPES, at least about 8 mM HEPES, at least about 9 mM HEPES, and at least about 10 mM HEPES. In some aspects, the buffer comprises at least about 5 mM HEPES.

In some aspects, the buffer comprises at least about 1 mM BICINE, at least about 2 mM BICINE, at least about 3 mM BICINE, at least about 4 mM BICINE, at least about 5 mM BICINE, at least about 6 mM BICINE, at least about 7 mM BICINE, at least about 8 mM BICINE, at least about 9 mM BICINE, and at least about 10 mM BICINE. In some aspects, the buffer comprises at least about 5 mM BICINE.

In some aspects, the buffer comprises at least about 1 mM CHES, at least about 2 mM CHES, at least about 3 mM CHES, at least about 4 mM CHES, at least about 5 mM CHES, at least about 6 mM CHES, at least about 7 mM CHES, at least about 8 mM CHES, at least about 9 mM CHES, and at least about 10 mM CHES. In some aspects, the buffer comprises at least about 5 mM CHES.

In some aspects, the buffer comprises at least about 1 mM CAPS, at least about 2 mM CAPS, at least about 3 mM CAPS, at least about 4 mM CAPS, at least about 5 mM CAPS, at least about 6 mM CAPS, at least about 7 mM CAPS, at least about 8 mM CAPS, at least about 9 mM CAPS, and at least about 10 mM CAPS. In some aspects, the buffer comprises at least about 5 mM CAPS.

In some aspects, the chromatography column comprises an AEX matrix, wherein the pH gradient mobile phase comprises about 5 mM 1,3 diaminopropane, about 5 mM diethanolamine, about 5 mM tris, about 5 mM imidazole, about 5 mM bis-tris, and about 5 mM piperazine, at pH of about 11.1. In some aspects, the chromatography column comprises an AEX matrix, wherein the pH gradient of the mobile phase comprises about 5 mM 1,3 diaminopropane, about 5 mM diethanolamine, about 5 mM tris, about 5 mM imidazole, about 5 mM bis-tris, about 5 mM piperazine, and about 5 mM acetic acid, at a pH of about 3.5.

In some aspects, the chromatography column comprises an CEX matrix, wherein the pH gradient mobile phase comprises about 5 mM malonic acid, about 5 mM formic acid, about 5 mM Acetic acid, about 5 mM MES, about 5 mM MOPSO, about 5 mM HEPES, about 5 mM BICINE, about 5 mM CHES, and about 5 mM CAPS, at a pH of about 4.0. In some aspects, the chromatography column comprises an CEX matrix, wherein the pH gradient mobile phase comprises about 5 mM malonic acid, about 5 mM formic acid, about 5 mM Acetic acid, about 5 mM MES, about 5 mM MOPSO, about 5 mM HEPES, about 5 mM BICINE, about 5 mM CHES, and about 5 mM CAPS, at a pH of about 11.0.

### B. Polypeptides

The methods disclosed herein can be used to isolate and/or purify a species of any polypeptide. In some aspects, the polypeptide is a protein. In some aspects, the species is a charge variant of the protein. In some aspects, the species is an acidic species. In some aspects, the species is a basic species. In some aspects, the species is the main species.

In some aspects, the protein has been subjected to a prior purification process before being subjected to the methods disclosed herein. In some aspects the protein has been subjected to, e.g., partially purified by, a prior affinity chromatography. In some aspects, the prior affinity chromatography comprises a protein A affinity chromatography.

In some aspects, the protein comprises a fusion protein. In some aspects, the protein comprises an immunoglobulin component fused to a biologically active polypeptide. In some aspects, the immunoglobulin component comprises a fragment of an antibody. In some aspects, the immunoglobulin component comprises a fragment of the constant region of an antibody. In some aspects, the immunoglobulin component comprises an Fc.

In some aspects, the protein comprises an immunoglobulin fused to a growth factor, a clotting factor, a cytokine, a chemokine, an enzyme, a hormone, or any combination thereof. In some aspects, the protein comprises an Fc fused to a CTLA-4 polypeptide. In some aspects, the protein comprises abatacept. In some aspects, the protein comprises belatacept. In some aspects, the protein comprises an Fc fused to an interleukin.

In some aspects, the protein comprises an antibody or an antigen-binding portion thereof. In some aspects, the antibody or antigen-binding portion thereof binds a tumor antigen. In some aspects, the antibody or antigen-binding portion thereof binds a checkpoint inhibitor. In some aspects, the antibody or antigen-binding portion thereof binds an antigen selected from PD-1, PD-L1, CTLA-4, LAG-3, TIGIT, GITR, CXCR4, CD73, HER2, VEGF, CD20, CD40, CD11a, tissue factor (TF), MICA/B PSCA, IL-8, EGFR, HER3, HER4, and any combination thereof.

In some aspects, the antibody or antigen-binding portion thereof specifically binds PD-1. Various human monoclonal antibodies that bind specifically to PD-1 with high affinity have been disclosed in U.S. Pat. Nos. 8,008,449, 6,808,710, 7,488,802, 8,168,757 and 8,354,509, US Publication No. 2016/0272708, and PCT Publication Nos. WO 2012/145493, WO 2008/156712,WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540, each of which is incorporated by reference in its entirety.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; also known as toripalimab; see Si-Yang Liu et al., J. Hematol. Oncol. 10:136 (2017)), BGB-A317 (Beigene; also known as Tislelizumab; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), and IBI308 (Innovent; see WO 2017/024465, WO 2017/025016, WO 2017/132825, and WO 2017/133540).

In one aspect, the anti-PD-1 antibody is nivolumab. In another aspect, the anti-PD-1 antibody is pembrolizumab.

In some aspects, the antibody or antigen-binding portion thereof specifically binds PD-L1. Examples of anti-PD-L1 antibodies include, but are not limited to, the antibodies disclosed in U.S. Pat. No. 9,580,507. In certain aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl):TPS3113 (2018)), and CK-301 (Checkpoint Therapeutics; see Gorelik et al., AACR:Abstract 4606 (April 2016)).

In certain aspects, the PD-L1 antibody is atezolizumab (TECENTRIQ®). In certain aspects, the PD-L1 antibody is durvalumab (IMFINZI™). In certain aspects, the PD-L1 antibody is avelumab (BAVENCIO®).

In some aspects, the antibody or antigen-binding portion thereof specifically binds CTLA-4. Human monoclonal antibodies that bind specifically to CTLA-4 with high affinity have been disclosed in U.S. Pat. No. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. In certain aspects, the CTLA-4 antibody is selected from the group consisting of ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2(3): 133-39 (2007)). In particular aspects, the anti-CTLA-4 antibody is ipilimumab. In particular aspects, the CTLA-4 antibody is tremelimumab. In particular aspects, the CTLA-4 antibody is MK-1308. In particular aspects, the CTLA-4 antibody is AGEN-1884.

In some aspects, the antibody or antigen-binding portion thereof specifically binds LAG-3. Antibodies that bind to LAG-3 have been disclosed in Int'l Publ. No. WO/2015/042246 and U.S. Publ. Nos. 2014/0093511 and 2011/0150892, each of which is incorporated by reference herein in its entirety. Non-limiting examples of anti-LAG-3 antibodies include but are not limited to 25F7 (described in U.S. Publ. No. 2011/0150892), BMS-986016, IMP731 (H5L7BW), MK-4280 (28G-10), REGN3767, humanized BAP050, IMP-701 (LAG-5250), TSR-033, BI754111,MGD013, or FS-118. These and other anti-LAG-3 antibodies useful in the claimed invention can be found in, for example: WO2016/028672, WO2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, and WO2014/140180, each of which is incorporated by reference herein in its entirety.

In some aspects, the antibody or antigen-binding portion thereof specifically binds CD137. Antibodies that bind to CD137 have been disclosed in U.S. Publ. No. 2005/0095244 and U.S. Pat. Nos. 7,288,638, 6,887,673, 7,214,493, 6,303,121, 6,569,997, 6,905,685, 6,355,476, 6,362,325, 6,974,863, and 6,210,669, each of which is incorporated by reference herein in its entirety. In some aspects, the anti-CD137 antibody is urelumab (BMS-663513), described in U.S. Pat. No. 7,288,638 (20H4.9-IgG4 [10C7 or BMS-663513]). In some aspects, the anti-CD137 antibody is BMS-663031 (20H4.9-IgG1), described in U.S. Pat. No. 7,288,638. In some aspects, the anti-CD137 antibody is 4E9 or BMS-554271, described in U.S. Pat. No. 6,887,673. In some aspects, the anti-CD137 antibody is an antibody disclosed in U.S. Pat. Nos. 7,214,493; 6,303,121; 6,569,997; 6,905,685; or 6,355,476. In some aspects, the anti-CD137 antibody is 1D8 or BMS-469492; 3H3 or BMS-469497; or 3E1, described in U.S. Pat. No. 6,362,325. In some aspects, the anti-CD137 antibody is an antibody disclosed in issued U.S. Pat. No. 6,974,863 (such as 53A2). In some aspects, the anti-CD137 antibody is an antibody disclosed in issued U.S. Pat. No. 6,210,669 (such as 1D8, 3B8, or 3E1). In some aspects, the antibody is Pfizer's PF-05082566 (PF-2566).

In some aspects, the antibody or antigen-binding portion thereof specifically binds KIR. Examples of anti-KIR antibodies have been disclosed in Int'l Publ. Nos. WO/2014/055648,WO 2005/003168, WO 2005/009465, WO 2006/072625, WO 2006/072626, WO 2007/042573,WO 2008/084106, WO 2010/065939, WO 2012/071411 and WO/2012/160448, each of which is incorporated by reference herein in its entirety. One anti-KIR antibody useful in the present disclosure is lirilumab (also referred to as BMS-986015, IPH2102, or the S241P variant of 1-7F9), first described in Int'l Publ. No. WO 2008/084106. An additional anti-KIR antibody useful in the present disclosure is 1-7F9 (also referred to as IPH2101), described in Int'l Publ. No. WO 2006/003179.

In some aspects, the antibody or antigen-binding portion thereof specifically binds GITR. Examples of anti-GITR antibodies have been disclosed in Int'l Publ. Nos. WO/2015/031667, WO2015/184,099, WO2015/026,684, WO11/028683 and WO/2006/105021, U.S. Pat. Nos. 7,812,135 and 8,388,967 and U.S. Publ. Nos. 2009/0136494, 2014/0220002, 2013/0183321 and 2014/0348841, each of which is incorporated by reference herein in its entirety. In one aspect, an anti-GITR antibody useful in the present disclosure is TRX518 (described in, for example, Schaer et al. Curr Opin Immunol. (2012) Apr; 24(2):217-224, and WO/2006/105021). In another aspect, the anti-GITR antibody is selected from MK4166, MK1248, and antibodies described in WO11/028683 and U.S. Pat. No. 8,709,424. In certain aspects, an anti-GITR antibody is an anti-GITR antibody that is disclosed in WO2015/031667. In certain aspects, an anti-GITR antibody is an anti-GITR antibody that is disclosed in WO2015/184099, e.g., antibody Hum231 #1 or Hum231 #2, or the CDRs thereof, or a derivative thereof (e.g., pab1967, pab1975 or pab1979). In certain aspects, an anti-GITR antibody is an anti-GITR antibody that is disclosed in JP2008278814, WO09/009116, WO2013/039954, US20140072566, US20140072565, US20140065152, or WO2015/026684, or is INBRX-110 (INHIBRx), LKZ-145 (Novartis), or MEDI-1873 (MedImmune). In certain aspects, an anti-GITR antibody is an anti-GITR antibody that is described in PCT/US2015/033991 (e.g., an antibody comprising the variable regions of 28F3, 18E10 or 19D3).

In some aspects, the antibody or antigen-binding portion thereof specifically binds TIM3. In some aspects, the anti-TIM3 antibody is selected from the anti-TIM3 antibodies disclosed in Int'l Publ. Nos. WO2018013818, WO/2015/117002 (e.g., MGB453, Novartis), WO/2016/161270 (e.g., TSR-022, Tesaro/AnaptysBio), WO2011155607, WO2016/144803 (e.g., STI-600, Sorrento Therapeutics), WO2016/071448, WO17055399; WO17055404, WO17178493, WO18036561, WO18039020 (e.g., Ly-3221367, Eli Lilly), WO2017205721, WO17079112; WO17079115; WO17079116, WO11159877, WO13006490, WO2016068802 WO2016068803, WO2016/111947, and WO/2017/031242, each of which is incorporated by reference herein in its entirety.

In some aspects, the antibody or antigen-binding portion thereof specifically binds OX40 (also known as CD134, TNFRSF4, ACT35 and/or TXGP1L). In some aspects, the anti-OX40 antibody is BMS-986178 (Bristol-Myers Squibb Company), described in Int'l Publ. No. WO20160196228. In some aspects, the anti-OX40 antibody is selected from the anti-OX40 antibodies described in Int'l Publ. Nos. WO95012673, WO199942585, WO14148895, WO15153513, WO15153514, WO13038191, WO16057667, WO03106498, WO12027328, WO13028231, WO16200836, WO 17063162, WO17134292, WO 17096179, WO 17096281, and WO 17096182, each of which is incorporated by reference herein in its entirety.

In some aspects, the antibody or antigen-binding portion thereof specifically binds NKG2A. In some aspects, the anti-NKG2A antibody is BMS-986315. In some aspects, the anti-NKG2A antibody is selected from the anti-NKG2A antibodies described in, for example, WO 2006/070286 (Innate Pharma S.A.; University of Genova); U.S. Pat. No. 8,993,319 (Innate Pharma S.A.; University of Genova); WO 2007/042573 (Innate Pharma S/A; Novo Nordisk A/S; University of Genova); U.S. Pat. No. 9,447,185 (Innate Pharma S/A; Novo Nordisk A/S; University of Genova); WO 2008/009545 (Novo Nordisk A/S); U.S. Pat. Nos. 8,206,709; 8,901,283; 9,683,041 (Novo Nordisk A/S); WO 2009/092805 (Novo Nordisk A/S); U.S. Pat. Nos. 8,796,427 and 9,422,368 (Novo Nordisk A/S); WO 2016/134371 (Ohio State Innovation Foundation); WO 2016/032334 (Janssen); WO 2016/041947 (Innate); WO 2016/041945(Academisch Ziekenhuis Leiden H.O.D.N. LUMC); WO 2016/041947 (Innate Pharma); and WO 2016/041945 (Innate Pharma), each of which is incorporated by reference herein in its entirety.

In some aspects, the antibody or antigen-binding portion thereof specifically binds ICOS In some aspects, the anti-ICOS antibody is BMS-986226. In some aspects, the anti-ICOS antibody is selected from anti-ICOS antibodies described in, for example, WO 2016/154177 (Jounce Therapeutics, Inc.), WO 2008/137915 (MedImmune), WO 2012/131004 (INSERM, French National Institute of Health and Medical Research), EP3147297 (INSERM, French National Institute of Health and Medical Research), WO 2011/041613 (Memorial Sloan Kettering Cancer Center), EP 2482849 (Memorial Sloan Kettering Cancer Center), WO 1999/15553 (Robert Koch Institute), U.S. Pat. Nos. 7,259,247 and 7,722,872 (Robert Kotch Institute); WO 1998/038216 (Japan Tobacco Inc.), U.S. Pat. Nos. 7,045,615; 7,112,655, and 8,389,690 (Japan Tobacco Inc.), U.S. Pat. Nos. 9,738,718 and 9,771,424 (GlaxoSmithKline), and WO 2017/220988 (Kymab Limited), each of which is incorporated by reference herein in its entirety.

In some aspects, the antibody or antigen-binding portion thereof specifically binds TIGIT. In some aspects, the anti-TIGIT antibody is BMS-986207. In some aspects, the anti-TIGIT antibody is clone 22G2, as described in WO 2016/106302. In some aspects, the anti-TIGIT antibody is MTIG7192A/RG6058/RO7092284, or clone 4.1D3, as described in WO 2017/053748. In some aspects, the anti-TIGIT antibody is selected from the anti-TIGIT antibodies described in, for example, WO 2016/106302 (Bristol-Myers Squibb Company) and WO 2017/053748 (Genentech).

In some aspects, the antibody or antigen-binding portion thereof specifically binds CSF1R. In some aspects, the anti-CSF1R antibody is an antibody species disclosed in any of international publications WO2013/132044, WO2009/026303, WO2011/140249, or WO2009/112245, such as cabiralizumab, RG7155 (emactuzumab), AMG820, SNDX 6352 (UCB 6352), CXIIG6, IMC-CS4, JNJ-40346527, MCS110, or the anti-CSF1R antibody in the methods is replaced with an anti-CSF1R inhibitor or anti-CSF1 inhibitor such as BLZ-945, pexidartinib (PLX3397, PLX108-01), AC-708, PLX-5622, PLX7486, ARRY-382, or PLX-73086.

### C. Methods of Treatment

Some aspects of the present disclosure are directed to methods of treating a subject comprising administering a species of a protein isolated and/or purified according to the methods disclosed herein. In some aspects, the subject has a tumor. In some aspects, the tumor is selected from a tumor derived from hepatocellular cancer, gastroesophageal cancer, melanoma, bladder cancer, lung cancer (e.g., NSCLC or SCLC), kidney cancer, renal cell carcinoma, head and neck cancer (e.g., a small cell carcinoma of the head and neck), colon cancer, prostate cancer, breast cancer, and any combination thereof. In some aspects, the tumor is relapsed or refractory. In some aspects, the tumor is locally advanced or metastatic.

### III. Compositions of the Disclosure

Some aspects of the present disclosure are directed to species of polypeptides, e.g., fusion proteins and/or antibodies, isolated and/or purified according to any method disclosed herein. In some aspects, the polypeptide is a protein. In some aspects, the species is a charge variant of the protein. In some aspects, the species is an acidic species of the protein. In some aspects, the species is a basic species of the protein. In some aspects, the species is the main species of the protein.

In some aspects, the protein comprises a fusion protein. In some aspects, the protein comprises an immunoglobulin component fused to a biologically active polypeptide. In some aspects, the immunoglobulin component comprises a fragment of an antibody. In some aspects, the immunoglobulin component comprises a fragment of the constant region of an antibody. In some aspects, the immunoglobulin component comprises an Fc.

In some aspects, the protein comprises an immunoglobulin fused to a growth factor, a clotting factor, a cytokine, a chemokine, an enzyme, a hormone, or any combination thereof. In some aspects, the protein comprises an Fc fused to a CTLA-4 polypeptide. In some aspects, the protein comprises abatacept. In some aspects, the protein comprises belatacept. In some aspects, the protein comprises an Fc fused to an interleukin.

In some aspects, the protein comprises an antibody or an antigen-binding portion thereof. In some aspects, the antibody or antigen-binding portion thereof binds a tumor antigen. In some aspects, the antibody or antigen-binding portion thereof binds a checkpoint inhibitor. In some aspects, the antibody or antigen-binding portion thereof binds an antigen selected from PD-1, PD-L1, CTLA-4, LAG-3, TIGIT, GITR, CXCR4, CD73, HER2, VEGF, CD20, CD40, CD11a, tissue factor (TF), MICA/B PSCA, IL-8, EGFR, HER3, HER4, and any combination thereof.

Various aspects of the disclosure are described in further detail in the following subsections. The present disclosure is further illustrated by the following examples which should not be construed as further limiting.

### EXAMPLES

### Example 1: Materials and Methods

The Examples described below use one or more of the following materials and methods.

### Equipment

For chromatographic separations, the following equipment were used: an ÄKTA™ Avant 25 chromatography system (FPLC) made by Cytiva—formerly known as GE Healthcare, an ALLIANCE E2695 (HPLC), coupled with a Fraction Collector III, both made by Waters Corporation, and a CONTICHROM® CUBE 30 (MCC) made by ChromaCon. The HPLC system was designed for analytical separations, and, when paired with a fraction collector, can provide high-resolution separation, but is very low-throughput. The FPLC system was designed for preparative chromatography applications, which provides low-resolution separation, but is much higher-throughput than the HPLC system. Finally, the MCC process provides equivalent separation to the FPLC system, but can utilize multiple (two) columns, as well as built-in methods (N-Rich), that allow for sample enrichment.

Where appropriate, the following analytical equipment were also used: iCE3™ system and Alcott 720 Autosampler made by Protein Simple, the DropSense96 made by Unchained Labs—formerly Trinean, and the ACQUITY UPLC™ system (Waters Corporation) coupled with a Maxis II™ quadruple-time-of-flight mass spectrometer made by Bruker Daltonics Inc.

### Chemicals and Materials

Four recombinant human monoclonal IgG antibodies (mAb1-3) were expressed in Chinese hamster ovary (CHO) cells and purified by affinity chromatography. Trypsin was purchased from Promega (Madison, WI, USA). Peptide N-glycosidase F enzyme (PNGase F) was obtained from New England Biolabs (Ipswich, MA, USA). All other reagents were purchased from Sigma-Aldrich (St. Louis, MO, USA) unless otherwise specified.

### Charge Variant Fractionation and Enrichment Using Liquid Chromatography

Charge variant fractionation using HPLC and FPLC:

Monoclonal antibody samples were injected onto MabPac SCX-10 (4 mm×250 mm) and MabPac SCX-10 (9 mm×250 mm) columns (Thermo Fisher Scientific, Wilmington, DE, USA) in Waters Alliance e2695 HPLC system and GE Avant 25 FPLC system, respectively. Maximum injection amount, by mass, based on the vendor's recommendation, was used to maximize separation productivity. Salt gradient mobile phases were 20 mM MES with and without 250 mM sodium chloride at pH 6.0, and 20 mM MES, pH 5.8 with and without 400 mM sodium chloride. Flow rate and elution gradient duration were optimized to achieve desirable separation. Fractions from HPLC system were collected using Waters Fraction Collector III. Fractions from FPLC were collected to Avant built-in fraction collector. Fractions were further pooled and characterized.

Charge variant enrichment using continuous chromatography:

Two Mono S CEX columns (10 mm×100 mm) and two Mono Q AEX columns (10 mm×100 mm) were purchased from Cytiva (Chicago, IL, USA). The column length of 100 mm was chosen to fit the pressure limit (<50 bar) of the continuous chromatography system when two columns were connected, in series, during continuous operation mode. Above-mentioned MES buffer and 50 mM Tris with and without 250 mM sodium chloride at pH 9.0 were used for CEX columns and AEX columns, respectively, for salt gradient elution. AEX pH gradient mobile phases were 5 mM 1,3 diaminopropane, 5 mM diethanolamine, 5 mM tris, 5 mM imidazole, 5 mM bis-tris, 5 mM piperazine, pH 11.1 and 5 mM 1,3 diaminopropane, 5 mM diethanolamine, 5 mM tris, 5 mM imidazole, 5 mM bis-tris, 5 mM piperazine, 5 mM acetic acid, pH 3.5. CEX pH gradient mobile phases were 5 mM malonic acid, 5 mM formic acid, 5 mM Acetic acid, 5 mM MES, 5 mM MOPSO, 5 mM HEPES, 5 mM BICINE, 5 mM CHES, 5 mM CAPS, pH 4.0 and 5 mM malonic acid, 5 mM formic acid, 5 mM Acetic acid, 5 mM MES, 5 mM MOPSO, 5 mM HEPES, 5 mM BICINE, 5 mM CHES, 5 mM CAPS, pH 11.0. Charge variant separation conditions in these columns were optimized using FPLC system before they were transferred to the Contichrom CUBE (ChromaCon) continuous chromatography system. A single run using transferred method was conducted in ChromIQ software. The region containing charge variant of interest was defined using the chromatography profile of the single run. After 10 cycles of enrichment and 2 cycles of depletion in CUBE system, fractions from CUBE system were collected using the external fraction collector for further pooling or analytical characterization.

### Charge Variant Sample Characterization

Imaged capillary isoelectric focusing (iCIEF):

To confirm the separation, iCIEF was performed using iCE3™ system and Alcott 720 Autosampler (Protein Simple) to quantify relative amounts of charge variants (acidic, main, and basic). The separation cartridge along with capillary was purchased from Convergent Bioscience. This capillary was fixed onto a glass substrate and separated from the catholyte and anolyte by two pieces of hollow fiber membrane. Samples were prepared by mixing protein at 2 g/L with a stock master mix solution containing relevant pI markers (Protein Simple), 1% methyl cellulose solution (Protein Simple), and PHARMALYTER® 3-10 (Cytiva), and urea, and diluting to 0.25 g/L with deionized water. After prepared samples were injected into the cartridge, a pre-focus period of 1-1.5 minutes at 1500 V and a focus period of 8-12 min at 3000 V were applied to achieve the optimum resolution. The final image of the IEF trace was captured by the 280 nm deuterium lamp detector. Results were processed by pI calibration using Protein Simple iCE Software while integration and quantification were calculated using Waters Empower3 software. Mass spectrometric characterization of the mAB fractions:

To identify root cause of charge variants, samples were analyzed as intact, after deglycosylation, and after proteolytic cleavage using trypsin.

Liquid chromatography—mass spectrometry (LC-MS) analysis of molecular masses of the intact and deglycosylated mAb:

Deglycosylated samples were prepared by mixing samples with PNGase F (New England Biolabs, Ipswich, MA) at 12.5 Unit/μg of protein at 37° C. for 1 hour.

An ACQUITY UPLC™ system (Waters Corporation, Milford, MA) coupled with a Maxis II™ quadruple-time-of-flight mass spectrometer (Bruker, Daltonics Inc., Billerica, MA) and a POROS™ R2/10 PERFUSION CHROMATOGRAPHY™ column (2.1 mm×100 mm, Thermo Scientific, Waltham, MA) were used to measure the molecular masses of the mAb samples. Throughout the analysis, the flow rate was set at 0.2 mL/min and the column temperature was set at 65° C. The sample was injected into the column at 90% mobile phase A (0.1% formic acid in LC-MS grade water) and 10% mobile phase B (0.1% formic acid in LC-MS grade acetonitrile). A linear gradient from 10% to 90% mobile phase B was used to elute the mAbs in 10 minutes.

The Maxis II mass spectrometer was controlled by the COMPASS HYSTAR™ software and run in positive mode with the following settings: a scan range of m/z 500-4000, gas temperature of 220° C., drying gas of 6 L/min, nebulizer at 2.5 Bar, and capillary voltage at 4500V. COMPASS DATAANALYSIS™, version 4.4, was used for mass spectrum deconvolution.

Peptide mapping using LC/MS/MS:

One hundred microliters of the enriched samples were denatured with 8 M guanidine hydrochloride (pH 8), reduced with 10 mM DTT at 37° C. for 20 min, and alkylated with 15 mM IAA at 37° C. for 20 min in darkness. The alkylated samples were buffer exchanged to digestion buffer (2 M Urea, 50 mM TRIS, and 10 mM CaCl₂, pH 7.6) by passing through Micro Bio-Spin 6 columns (Bio-Rad, Hercules, California). The eluates were enzymatically digested at 37° C. for 3 hours with trypsin using a 1:25 (w/w, enzyme/protein) ratio. Upon completion of digestion, the digested samples were acidified with addition of 1 N hydrochloric acid.

The tryptic digest was chromatographically separated using a Waters ACQUITY UPLC™ system (Milford, MA, USA) before being analyzed by Thermo Scientific ORBITRAP Q-EXACTIVE™ PLUS mass spectrometer (Thermo Scientific, Bremen, Germany). A Waters Acquity BEH C18 column (1.7 μm, 2.1×150 mm) was used for separation at 45° C. with 0.1% formic acid in water as mobile phase A and 0.1% formic acid in acetonitrile as mobile phase B. A linear gradient of 1% to 80% mobile phase B over 105 minutes was used to elute the peptides at a flow rate of 0.2 mL/min. The Q EXACTIVE PLUS™ mass spectrometer was operating in data dependent mode to switch between MS and MS/MS acquisition. Ions were generated using a sheath gas flow rate of 40 units, an auxiliary gas flow rate of 10 units, a spray voltage of 3 kV, a capillary temperature of 275° C., and an S-Lens RF level of 60 units. Resolution was set at 70,000 (AGC target 3e6) and 17,500 (AGC target 1e5) for survey scans and MS/MS events, respectively. The dynamic exclusion duration of 10 seconds was used with a single repeat count. The mass spectrometry data analysis was facilitated using THERMO PROTEOME DISCOVERER™ software package, version 1.4 (Thermo Scientific, Bremen, Germany).

### Example 2: Enrichment Using Multi-Column Continuous Chromatography Methodology

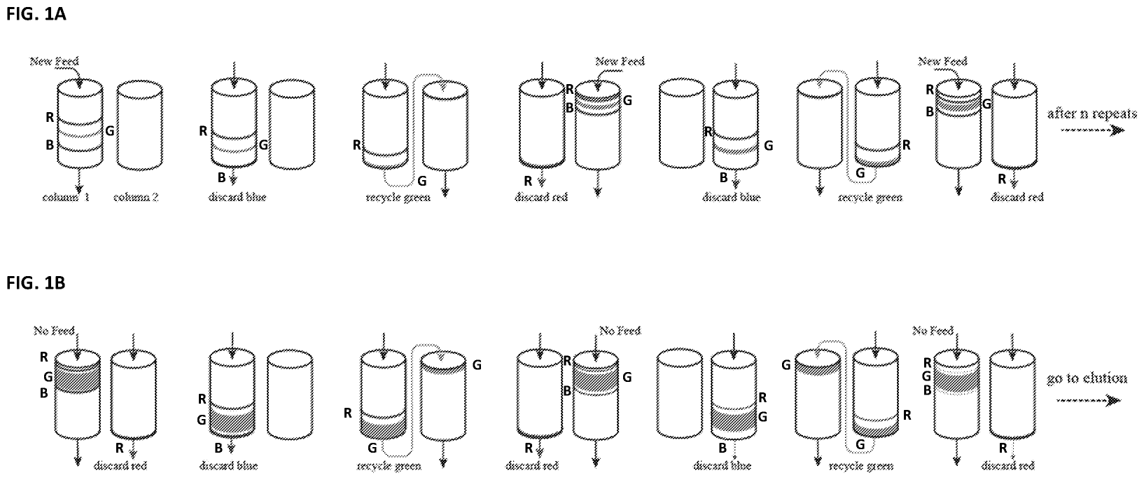

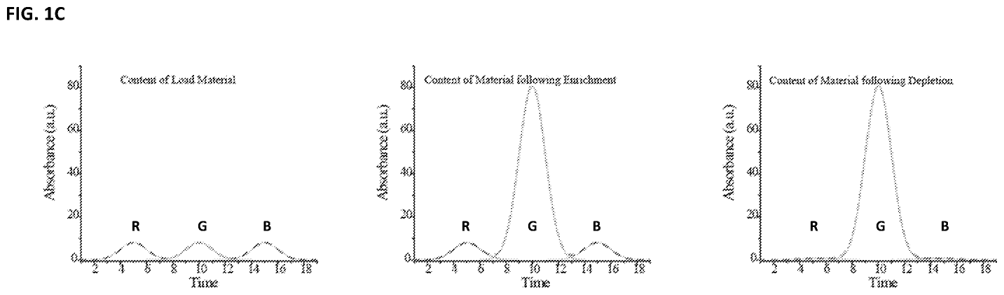

As described herein, the enrichment methods provided herein generally involve the use of two identical (twin) columns and MCSGP methodology. A schematic of the overall process is outlined in FIGS. 1A-1B. As shown, enrichment is achieved through a three stage operation including enrichment (of green—i.e., species of interest), depletion (of red and blue—i.e., species of non-interest), and elution (of green). Firstly, a switch is referred to as a run on a single column, whereas a cycle refers to a run on each of the two columns, and is equivalent to two switches. The feed material is represented by a mixture of three components, or species, shown in red, green, and blue, with the green species representing the species of interest for enrichment.

During the enrichment stage (see FIG. 1A), the feed material is loaded onto the first column. After discarding the species that elutes from the column first (blue), the species for enrichment (green) is recycled to the second column by connecting the inlet of column 2 to the outlet of column 1. After all of the species for enrichment (green) is recycled from the first column to the second column, the later eluting species (red) is discarded from the first column via a strip step, and then column 1 is re-equilibrated. During column 1 re-equilibration, additional load (mixture) is injected to the column 2 to join the green species that was recycled from column 1. The same separation operation from column 1 (discarding blue, recycling green, and discarding red) is then conducted on column 2. The desired species (green) is enriched as the number of cycles increases. During the depletion stage (see FIG. 1B), the separation cycling pattern that was done in the enrichment stage is performed again, except that there is no additional load material added to the columns after a recycle has been completed. This stage consists of 1 cycle, meaning one separation on each of the two columns. The species of interest (green) stays in the column and the unwanted species (blue and red) are removed for the system as depletion occurs. Depletion is followed by stage 3, called elution, to recover the green species from the system. The elution phase is an elongated version of the separation gradient that was used to enrich the green species, where fractionation of the remaining product can be implemented.

FIG. 1C provides the results of a simulation of the UV trace was performed using the enrichment method outlined above. The left panel shows the presence of the three species (red, blue, and green) present in the initial feed material. The middle panel shows the resulting product following 10 cycles of enrichment of the green species. The right panel shows that after 1 cycle of depletion, there was virtually none of the species of non-interest (i.e., blue and red species) left in the system, while the species of interest (i.e., green) remain unaltered. As demonstrated herein, the species of interest was eluted during the final stage of processing, and collected for subsequent analysis.

### Example 3: Comparison of Batch and Multi-Column Continuous Chromatography Methods for Charge Variant Enrichment

In order to assess the capability of the enrichment methods provided herein (e.g., continuous chromatography MCSGP), HPLC, FPLC, and continuous chromatography (CUBE) were used to separate the acidic variants of a recombinant human IgG antibody (mAb1). The specific methods used are provided in Example 1.

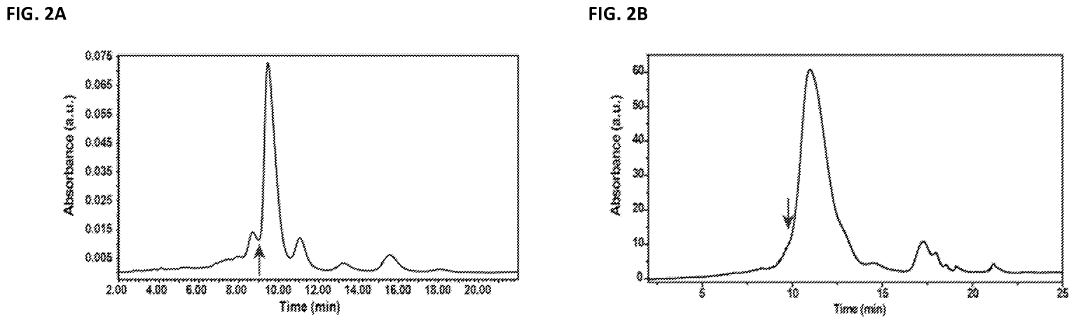

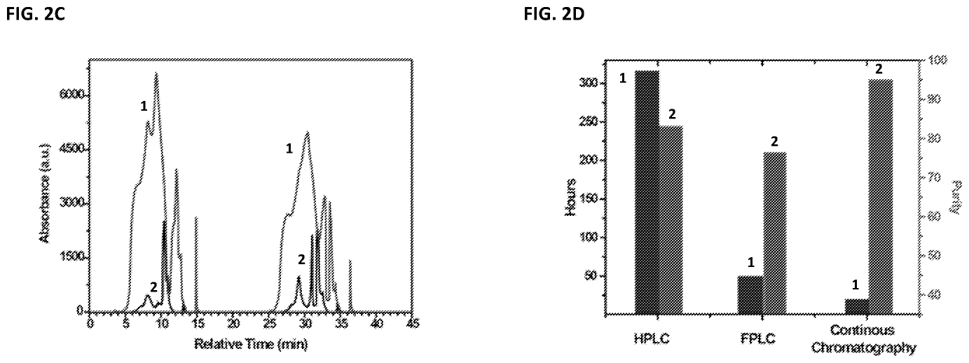

As shown in FIGS. 2A and 2B, the separation pattern using HPLC (FIG. 2A) and FPLC (FIG. 2B) were generally similar with the acidic variants present in the early-eluting fractions that were collected (prior to the arrow). As between HPLC and FPLC, improved resolution was observed with HPLC. But a benefit of the FPLC compared to HPLC is that a greater amount of the initial material was able to be processed (injection amount was 10 times or more). FIG. 2C provides the separation profile using the CUBE system. As shown, only the separation of the acidic species was visible on the chromatogram, as the enrichment method discarded any materials that were not targeted for enrichment. Like with FPLC, the CUBE system allowed for the processing of much greater amount of material (i.e., larger quantity of the material was able to be loaded to the CUBE system per injection). As to resolution, it appears to be between what was achieved using the HPLC and FPLC systems. As shown in FIG. 2C, a key benefit of the enrichment method using the CUBE system was that after 10 cycles of enrichment (red) the acidic peak was significantly greater than that observed after 2 cycles (black), demonstrating the ability of the CUBE system to highly enrich for species of interest (acidic variant). Following depletion, implementation of fractionation was an option for the elution phase that allowed for flexible pooling of the product.

From the data generated using these three different methodologies, projections for the time required in order to generate 10 mg of acidic variant from a load sample containing 17% acidic variant content were estimated. As shown in FIG. 2D, the estimations were 300, 48, and 10 hours, and the resulting purity (or acidic percentage) determined using an iCIEF method of the samples were 87%, 78%, and 95%, for the HPLC, FPLC and CUBE systems, respectively. These results confirmed that the continuous chromatography enrichment methods provided herein was able to achieve the highest purity in the shortest amount of time.

Another important property of the enrichment methods provided herein is the capability to separate sub-species of variants in enriched acidic or basic peak regions using elution fractionation. To further assess this aspect, two continuous chromatography enrichment runs (one run focusing on the acidic peak region, and the other focusing on basic peak region) were performed to separate the different variants of the mAb1 antibody.

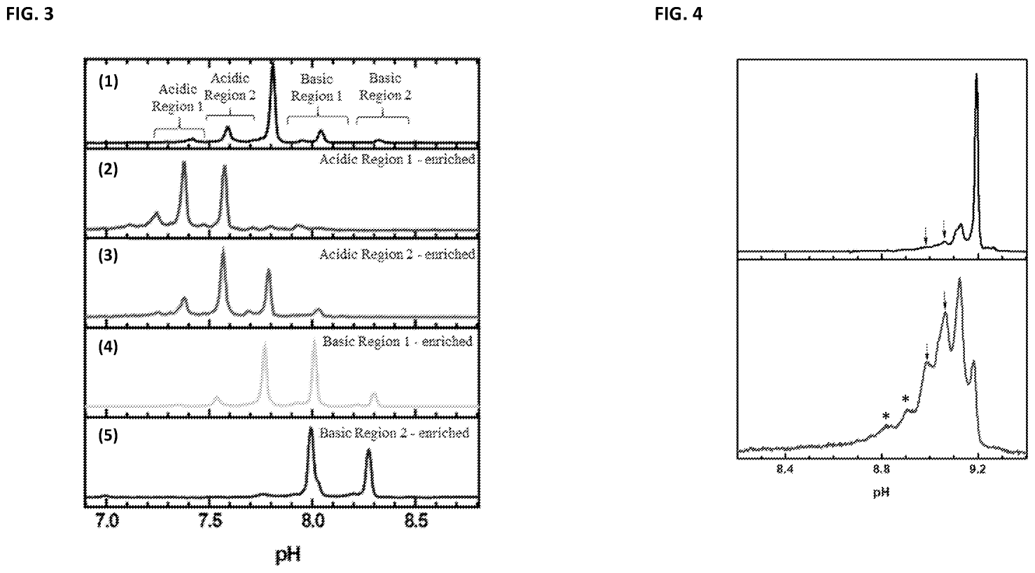

As shown in FIG. 3 (panel 1), for each of the runs, two peaks were fractionated: region 1 and region 2. As shown in panels 2 and 3, acidic variants were enriched in both acidic regions 1 and 2, with acidic region 1 containing more acidic variants. Additionally, the acidic variant distributions differed for acidic regions 1 and 2. Acidic region 1 also contained more acidic variants containing two extra negative charges (migrating to a pI of ˜7.2 and ˜7.4, respectively), which were almost not detectable in the load material (see panel 1 of FIG. 3). As shown in panels 4 and 5, similar separation efficiency and patters were observed in the basic region enrichment. These results demonstrate that by combining this enrichment of a particular peak region and further fractionation of the elution peak, the enrichment methods provided herein allow for the identification of specific charge variants present in a given sample.

### Example 4: Comparison of Separation Conditions When Enriching Charge Variants

To further assess the different separation/enrichment conditions, both CEX and AEX columns where used with salt gradient and with pH gradient to separate the acidic and basic charge variants of the mAb3 antibody.

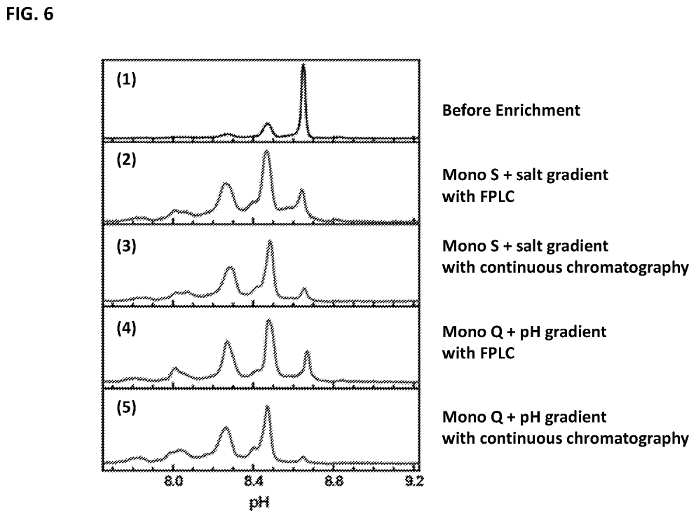

As shown in FIG. 6, the best separation results were observed for CEX (Mono S) when used with a salt gradient. For AEX (Mono Q), the best separation results were observed with the pH gradient. Using these optimized separation conditions, samples from the FPLC fractionation runs and the enriched samples from continuous chromatography runs were collected for further analysis using iCIEF and MS. As shown with the iCIEF data provided in FIG. 6, much improved enrichment was observed using the continuous chromatography method (panels 3 and 5) compared to the FPLC fractionation method using batch-mode separation (panels 2 and 4).

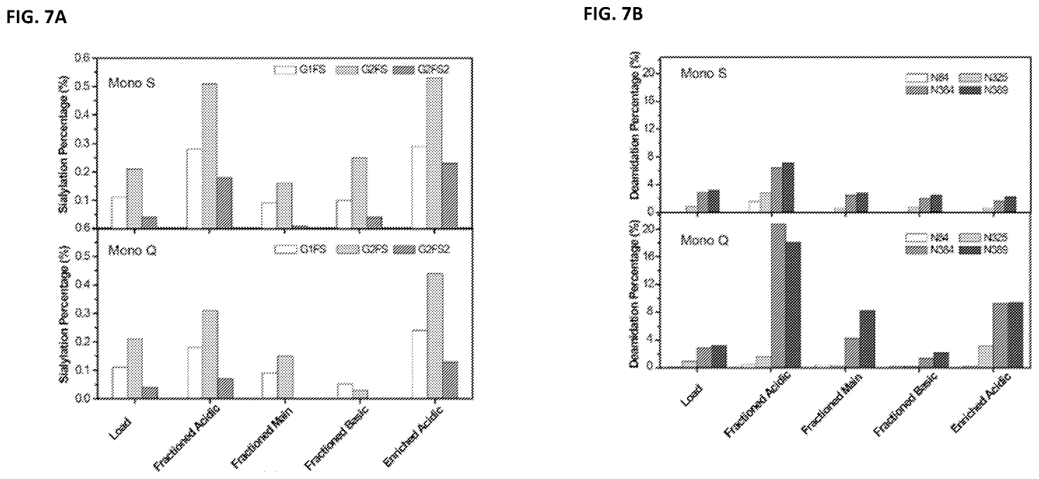

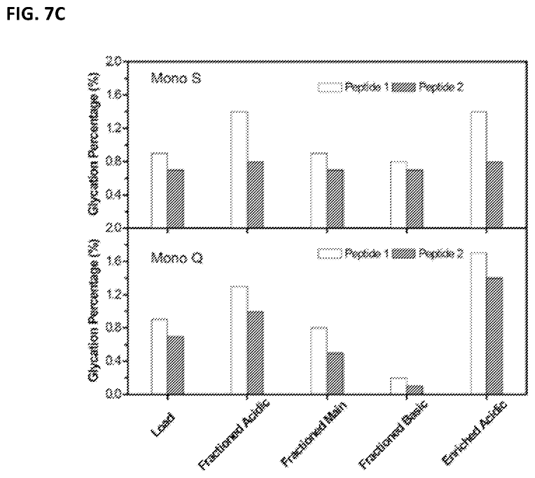

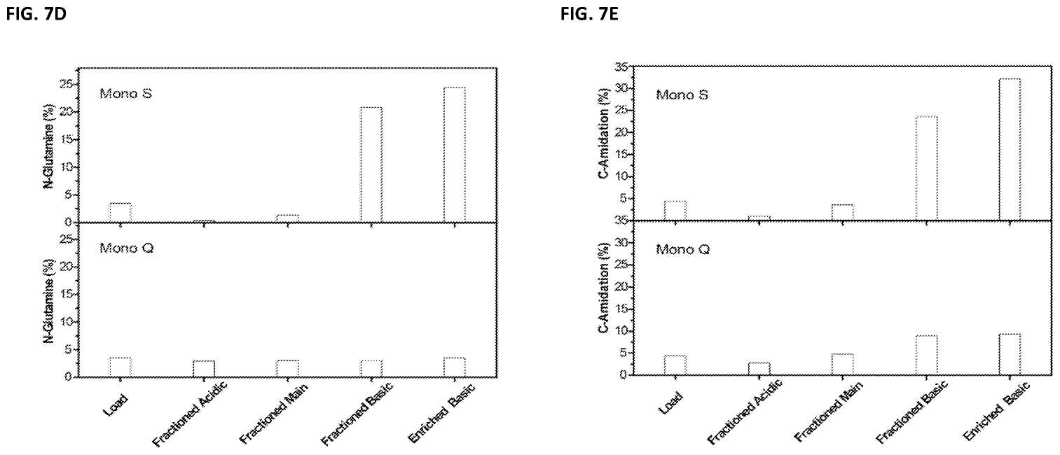

Comparison of iCIEF profiles alone using CEX with salt gradient (panel 3) and AEX with pH gradient (panel 5), it was not possible to visually distinguish the difference in separation efficiency between these methods. But with LC/MS peptide mapping, several acidic variants including glycan sialylation, deamidation, and glycation were observed (see FIGS. 7A, 7B, and 7C). Interestingly, as shown, the two different methods described above resulted in enrichment of different acidic variants to different extents based on MS analysis. Not to be bound by any theory, this could be due to the fact that the separation was not solely dependent on charge interactions during ion exchange chromatography.

As shown in FIGS. 7B and 7C, the use of AEX with a pH gradient resulted in more effective separation of deamidated and glycated (acidic) species compared to using CEX with salt gradient. The deamidation level at different Asn sites (including N84, N325, N384, and N389) showed very small differences in acidic, main, and basic fractions from CEX column with salt gradient. FIG. 7B (top panel). The observed deamidation levels at N384 and N389 in the PENNY loop were no higher than 7% even in the acidic fractions. However, the deamidated species were significantly enriched in the acidic fractions from AEX column with pH gradient. The deamidation levels for N384 and N389 were about 20% in the acidic sample collected from FPLC and about 9% in the acidic samples collected from CUBE (FIG. 7B, bottom panel).

As shown in FIG. 7C, a similar trend was observed in the glycation levels in two identified peptides, suggesting more effective enrichment of glycated species using an AEX columns with a pH gradient (Mono Q). In contrast, as shown in FIGS. 7D and 7E, CEX with a salt gradient (Mono S) was more effective in enriching for basic species containing un-cyclized N-terminal glutamine and C-terminal proline amidation. Both N-terminal glutamine and C-terminal lysine in all fractions from AEX were present at similar levels. However, N-terminal glutamine and C-terminal lysine were detected in the basic fractions at a much higher level, as shown in top panels in FIGS. 7D and 7E. In general, comparing with the fractionated samples from FPLC system, the enriched samples collected from the CUBE system contained the same or higher levels of targeted species, except deamidated species.

Collectively, the above results demonstrate that sample generation using the enrichment methods provided herein (MCSGP methodology) was highly effective and efficient, facilitating the studies to establish structure and function relationships.

### Example 5: Impact of Improved Sample Purity on Analytical Characterization

As demonstrated in the above Examples, the enrichment methods provided herein allowed for the identification of species that were not as clearly detectable either in the initial load sample in samples generated using FPLC. Additional benefits of the enrichment methods provided herein are further highlighted below.

### ICIEF

Comparison of image capillary electrophoresis profiles before and after enrichment of the acidic region of a mAb2 antibody sample is provided in FIG. 4. Following enrichment, two shoulder signals present prior to the enrichment became much more defined peak signals following enrichment (see arrows in top and panels of FIG. 4). Additionally, two acidic peaks that were undetectable in the load sample prior to the enrichment were observed (see * in bottom panel of FIG. 4).

### Mass Spectrometry

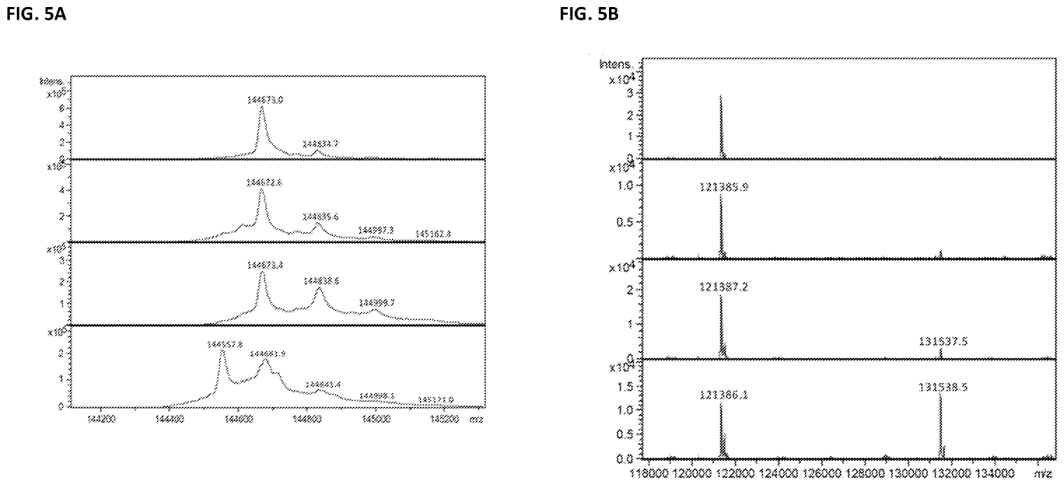

As described herein, the enrichment methods provided herein were not only able to enrich charge variants that were observed in the initial sample but also allowed for the identification of additional variants that were not detectable previously. As shown in FIG. 5A, glycation signals from deglycosylated LC-MS assay became stronger in the enriched acidic sample (third panel from the top). Additionally, in the acidic variant separation of mAb1, a new peak (the left-most peak in panel 2 of FIG. 3) was detected in the sample of enriched acidic region 1. Similar new peaks were observed in acidic variant separation of mAb2 (see * in bottom panel of FIG. 4).

Based on MS analysis, a new peak (around 144557.8 m/z) was also observed in the enriched basic variant sample (the left peak in the bottom panel of FIG. 5A). This peak was attributed to the species with proline amidation after C-terminal glycine removal. As shown in FIG. 5B (bottom panel), the peak around 131538 m/z was only clearly observed in the enriched acidic variant sample. The peak was assigned to heavy chain truncated species with one heavy chain missing residues after 330. Without the use of the continuous chromatography enrichment methods provided herein, the detection of such peaks would not have been possible, especially in such a short time frame.

In addition to the above and as described below, the enrichment methods provided herein also resulted in improved detection of certain post-translational modifications (PTMs). The results of all the PTMs that were identified in the fractionated and enriched samples of different recombinant human monoclonal IgG antibodies are provided, e.g., in FIG. 3 (mAb1), FIG. 4 (mAb2), and FIGS. 5A-5B and 6 (mAb3).

### Sialic Acid

As shown in FIG. 5A, MS analysis showed that using the enrichment methods provided herein, an elevated level of Fc-sialylation in acidic fractions, particularly in the enriched acidic fraction of mAb3. The MS analysis further suggested that the detected sialylation occurred at the end of the N-glycans (G1F and G2F) (FIG. 7A).

### Demaidation and Aspartic Acid Isomerization

Asn deamidation was observed on residues in both the CDR and constant region of the antibodies. The two most predominate deamidated residues were N384 and N389. N384 was followed by Gly residue, and N389 was in the PENNY motif. NG and PENNY motifs were previously reported as deamidation hot-spots. The other two detected deamidation sites were N84 and N325, and were followed by Ser and Lys, respectively (FIG. 7B).

### Glycation

Using the enrichment methods provided herein, glycation of peptides located in the variable region, close to the N-terminus of the mAb, was also observed. As shown in FIG. 7C, the identification of glycation in the fractions generated from a CEX column using a salt gradient was different than that of the samples generated using an AEX column and a pH gradient. There was more variability in the samples generated using the AEX column with a pH gradient, indicating that this separation method provided better resolution for this charge variant species.

### N-Terminal Glutamine Modification

A high percentage of N-glutamine was also detected in the basic fractions (FIG. 7D). The continuous chromatography enrichment showed better separation compared to batch mode fractionation. The use of Mono S (CEX) with salt gradient resulted in enhanced detection of pyroglutamation. This PTM was not expected to have impact on product activity for two reasons: (1) the first residue was not in antigen binding interface, and (2) non-pyrolated N-terminus would be converted to pyrolated one once the product was injected into human body because of the presence of glutaminyl cyclase.

### C-Terminal Lysine

Basic variants of the mAb1 antibody were enriched using a Mono Q (AEX) column and a pH gradient (panels 4 and 5 of FIG. 3). The subsequent analysis of these enriched basic species, following treatment with carboxypeptidase B (CpB), confirmed the presence of C-terminal Lys. Differences in antigen affinity for these variants were minimal because the complementarity-determining region (CDR) was at the opposite end of mAb to where the Lys variants was found.

### C-Terminal Proline Amidation

From the LC-MS data, a C-terminal amidation modification in mAb3 basic species was observed using a Mono S (CEX) column with a salt gradient. Enrichment of this sample using the methods provided herein (MCSGP) increased the relative content of this modification in the basic species sample 6-fold over the load material, and 20% over the FPLC fractionated sample (FIG. 7E).

### Truncated Species

As shown in FIG. 5B, using the enrichment methods provided herein, a truncated species at 131538 Da in deglycosylated was observed using MS analysis. This species was attributed to a truncation leading to a loss of residues from 330 to 446 in one of the heavy chains. The cutting site (P329) was in the loop region before the last beta strand in CH2 domain. Additionally, a truncated species of 97.5 kDa in an acidic fraction was also observed. This truncated species was attributed to a heavy chain fragment, from the hinge region to its end.

Collectively, the results provided herein demonstrate that compared to traditional batch mode methods, the continuous chromatography enrichment methods of the present disclosure (MCSGP) allows for enrichment of target charge species with both high productivity and high purity. Using samples enriched with the continuous chromatography provides significant benefits for analytical characterization.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein.

All publications, patent applications, patents, and other references mentioned herein are incorporated by reference in their entirety. Database entries and electronic publications disclosed in the present disclosure are incorporated by reference in their entireties. The version of the database entry or electronic publication incorporated by reference in the present application is the most recent version of the database entry or electronic publication that was publicly available at the time the present application was filed. The database entries corresponding to gene or protein identifiers (e.g., genes or proteins identified by an accession number or database identifier of a public database such as Genbank, Refseq, or Uniprot) disclosed in the present application are incorporated by reference in their entireties. The gene or protein-related incorporated information is not limited to the sequence data contained in the database entry. The information incorporated by reference includes the entire contents of the database entry in the most recent version of the database that was publicly available at the time the present application was filed. In case of conflict, the present specification, including definitions, will control. In addition, the materials, methods, and examples are illustrative only and not intended to be limiting.

1. A method of isolating a species of a protein from a mixture comprising the species and one or more impurities, comprising:
(a) contacting the mixture with two or more chromatography columns in a continuous operation mode in a chromatographic separation system; and
(b) separating the species of the protein.

2. A method of increasing purity and/or yield of a species of a protein from a mixture comprising the species and one or more impurities, comprising:
(a) contacting the mixture with two or more chromatography columns in a continuous operation mode in a chromatographic separation system; and
(b) separating the species of the protein, thereby increasing purity and/or yield of the species.

3. The method of claim 2, further comprising subjecting the species from (a) to analytical characterization.

4. A method for conducting analytical characterization of a species of a protein, comprising:
(a) separating the species of the protein from a mixture comprising the species and one or more impurities, comprising contacting the mixture with two or more chromatography columns in a continuous operation mode in a chromatographic separation system; and
(b) conducting analytical characterization of the species from (a).

5. The method of claim 3, wherein the analytical characterization is conducted by a HPLC system, capillary isoelectric focusing (cIEF) gel electrophoresis, Imaged Capillary Isoelectric Focusing (iCIEF), cation exchange chromatography (CEX), anion exchange chromatography (AEX), MFI, SEC-MALS, SEC, or mass spectrometry.

6. The method of claim 2, which yields an increased purity and/or an increased yield of the species of the protein compared to an HPLC or an FPLC.

7. (canceled)

8. The method of claim 2, comprising loading the mixture to a first chromatography column and a second chromatography.

9. The method of claim 8, wherein the loaded mixture passes through the first chromatography column and is separated into an enrichment species comprising the species and a discard species (“enrichment stage I”).

10. The method of claim 9, wherein the enrichment species passes through the second column and the discard species is discarded after the first chromatography column (“enrichment stage II”).

11. The method of claim 10, further comprising re-equilibrating the first chromatography column.

12. The method of claim 10, further comprising contacting the enrichment species with the first chromatography column.

13. The method of claim 8, further comprising loading an additional mixture to the first column, wherein the additional mixture comprises the species and one or more impurities.

14. The method of claim 13, wherein the additional mixture is added at the same time the enrichment species is added to the first chromatography column.

15. The method of claim 13, wherein the additional mixture is added after the enrichment species is added to the first chromatography column, and before the enrichment species passes through the first chromatography column.

16. The method of claim 13, wherein the enrichment stages I and II are repeated at least twice, at least three times, at least four times, at least five times, at least six times, at least seven times, at least eight times, at least nine times, or at least ten times.

17. The method of claim 16, further comprising a depletion stage.

18. The method of claim 17, wherein the depletion stage comprises contacting the enrichment species with the first chromatography column in the absence of an additional mixture.

19. The method of claim 18, wherein the depletion stage further comprises passing the enrichment species through the first chromatography column and separating the species from one or more impurities.

20. The method of claim 19, wherein the depletion stage further comprises passing the enrichment species through the second chromatography column, separating the species from one or more impurities.

21. The method of claim 2, further comprising eluting the species.

22-23. (canceled)

24. The method of claim 2, wherein one or more of the chromatography columns comprise a salt gradient, a pH gradient, or both.

25. The method of claim 24, wherein the salt gradient comprises a sodium chloride gradient.

26. The method of claim 24, wherein the salt gradient comprises with or without the salt.

27-28. (canceled)

29. The method of claim 24, wherein the pH of the pH gradient is between about pH 3 and about pH 11.

30. The method of claim 2, wherein the mixture is in a buffer.

31. (canceled)

32. The method of claim 2, further comprising measuring a post-translational modification.

33. (canceled)

34. The method of claim 2, wherein the protein comprises a fusion protein or an antibody or antigen binding portion thereof.

35-40. (canceled)

41. The method of claim 34, wherein the species of the fusion protein or antibody is an acidic species, a basic species or a main species.

42. The method of claim 34, wherein the fusion protein or antibody is partially purified by protein A affinity chromatography.

43. The method of claim 2, wherein the species is separated by a counter current purification system.

44-47. (canceled)
